(19) United States
(12) Patent Application Publication
(10) Pub. No.: US 2008/0280836 A1
(43) Pub. Date: Nov. 13, 2008

(54) Anti-hypercholesterolemic biaryl azetidinone compounds

(76) Inventors: Gregori J. Morriello, Randolph, NJ (US); Robert J. DeVita, Westfield, NJ (US)

Correspondence Address: MERCK AND CO., INC, P O BOX 2000, RAHWAY, NJ, 07065-0907, US

(21) Appl. No.: 12/151,647

(22) Filed: May 8, 2008

Related U.S. Application Data
(60) Provisional application No. 60/928,214, filed on May 8, 2007.

(51) Int. Cl. A61K 31/7052; C07D 205/04; A61K 31/397; A61P 3/00; C07H 7/06

(52) U.S. Cl. 514/23; 540/200; 514/210.02; 536/53

(57) ABSTRACT

This invention provides cholesterol absorption inhibitors of Formula I:

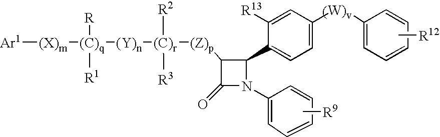

and the pharmaceutically acceptable salts thereof, wherein R¹² is an alkyl, alkeny or alkynyl group mono- or poly-substituted with —OH, —COOH or a combination of —OH and —COOH, and R⁹ contains an alkyl, alkeny or alkynyl group substituted with a heterocyclic ring, amino or sulfonyl. The compounds are useful for lowering plasma cholesterol levels, particularly LDL cholesterol, and for treating atherosclerosis and preventing atherosclerotic disease events.

### BACKGROUND OF THE INVENTION

The instant invention relates to substituted 2-azetidinones and the pharmaceutically acceptable salts there of, and to their use alone or in combination with other active agents to treat hypercholesterolemia and for preventing, halting or slowing the progression of atherosclerosis and related conditions and disease events.

It has been clear for several decades that elevated blood cholesterol is a major risk factor for coronary heart disease, and many studies have shown that the risk of CHD events can be reduced by lipid-lowering therapy. Prior to 1987, the lipid-lowering armamentarium was limited essentially to a low saturated fat and cholesterol diet, the bile acid sequestrants (cholestyramine and colestipol), nicotinic acid (niacin), the fibrates and probucol. Unfortunately, all of these treatments have limited efficacy or tolerability, or both. Substantial reductions in LDL (low density lipoprotein) cholesterol accompanied by increases in HDL (high density lipoprotein) cholesterol could be achieved by the combination of a lipid-lowering diet and a bile acid sequestrant, with or without the addition of nicotinic acid. However, this therapy is not easy to administer or tolerate and was therefore often unsuccessful except in specialist lipid clinics. The fibrates produce a moderate reduction in LDL cholesterol accompanied by increased HDL cholesterol and a substantial reduction in triglycerides, and because they are well tolerated these drugs have been more widely used. Probucol produces only a small reduction in LDL cholesterol and also reduces HDL cholesterol, which, because of the strong inverse relationship between HDL cholesterol level and CHD risk, is generally considered undesirable. With the introduction of lovastatin, the first inhibitor of HMG-CoA reductase to become available for prescription in 1987, for the first time physicians were able to obtain large reductions in plasma cholesterol with very few adverse effects.

Studies have unequivocally demonstrated that lovastatin, simvastatin and pravastatin, all members of the HMG-CoA reductase inhibitor class, slow the progression of atherosclerotic lesions in the coronary and carotid arteries. Simvastatin and pravastatin have also been shown to reduce the risk of coronary heart disease events, and in the case of simvastatin a highly significant reduction in the risk of coronary death and total mortality has been shown by the Scandinavian Simvastatin Survival Study. This study also provided some evidence for a reduction in cerebrovascular events. Despite the substantial reduction in the risk of coronary morbidity and mortality achieved by simvastatin, the risk is still substantial in the treated patients. For example, in the Scandinavian Simvastatin Survival Study, the 42% reduction in the risk of coronary death still left 5% of the treated patients to die of their disease over the course of this 5 year study. Further reduction of risk is clearly needed.

A more recent class of anti-hyperlipidemic agents that has emerged includes inhibitors of cholesterol absorption. Ezetimibe, the first compound to receive regulatory approval in this class, is currently marketed in the U.S. under the tradename ZETIA®. Ezetimibe has the following chemical structure and is described in U.S. Pat. Nos. Re. 37721 and 5,846,966:

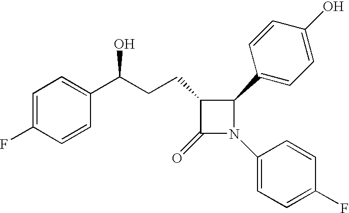

Sugar-substituted 2-azetidinones, including glucuronidated analogs of the following general structure:

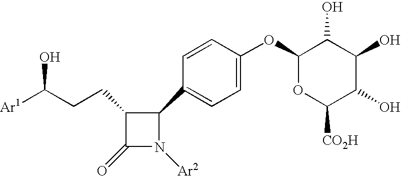

and methods for making them are disclosed in U.S. Pat. No. 5,756,470, wherein Ar¹ and Ar² are unsubstituted or substituted aryl groups.

Additional cholesterol absorption inhibitors are described in WO2002/066464 A1 (applied for by Kotobuki Pharmaceutical Co.), and US2002/0137689 A1 (Glombik et al.). WO2002/066464 A1 discloses hypolipidemic compounds of general formula

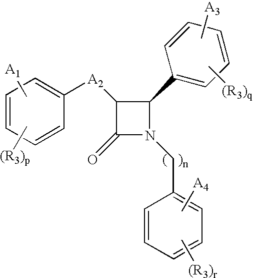

wherein, among other definitions, A₁, A₃ and A₄ can be

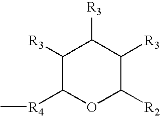

and wherein R₂ is —CH₂OH, —CH₂OC(O)—R₁, or —CO₂R₁; R₃ is —OH or —OC(O)R₁, and R₄ is —(CH₂)ₖR₅(CH₂)ᵢ— where k and i are zero or integers of one or more, and k+i is an integer of 10 or less; and R₅ is a single bond, —CH═CH—, —OCH₂—, carbonyl or —CH(OH).

US2002/0137689 A1 discloses hypolipidemic compounds of general formula

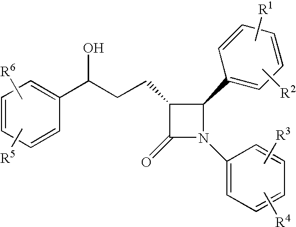

wherein, among other definitions, R¹, R², R³, R⁴, R⁵, R⁶ independently of one another can be (C₀-C₃₀)-alkylene-(LAG), where one or more carbon atoms of the alkylene radical may be replaced by —O—, —(C═O)—, —CH═CH—, —C≡C—, —N((C₁-C₆)-alkyl)-, —N((C₁-C₆)-alkylphenyl) or —NH—; and (LAG) is a sugar residue, disugar residue, trisugar residue, tetrasugar residue; a sugar acid, or an amino sugar.

Additionally, PCT publication WO2005/047248, published May 26, 2005, (applied for by Microbia, Inc.) discloses 4-biarylyl-1-phenylazetidin-2-ones for the treatment of hypercholesterolemia, having the general formula:

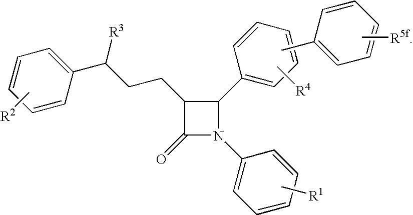

In the ongoing effort to discover novel treatments for hyperlipidemia and atherosclerotic process, the instant invention provides novel cholesterol absorption inhibitors, described below.

### SUMMARY OF THE INVENTION

One object of the instant invention is to provide novel cholesterol absorption inhibitors of Formula I

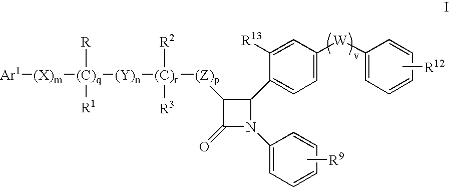

and the pharmaceutically acceptable salts thereof.

A second object of the instant invention is to provide a method for inhibiting cholesterol absorption comprising administering a therapeutically effective amount of a compound of Formula I to a patient in need of such treatment. Another object is to provide a method for reducing plasma cholesterol levels, especially LDL-cholesterol, and treating hypercholesterolemia comprising administering a therapeutically effective amount of a compound of Formula I to a patient in need of such treatment.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a patient who is at risk of developing atherosclerosis or who already has atherosclerotic disease. Another object of the present invention is the use of the compounds of the present invention for the manufacture of a medicament useful in treating, preventing or reducing the risk of developing these conditions. Other objects of this invention are to provide processes for making the compounds of Formula I and to provide novel pharmaceutical compositions comprising these compounds.

Additionally the compounds of this invention, particularly radioactive isotopes of the compounds of Formula I, can be used in screening assays, where the assay is designed to identify new cholesterol absorption inhibitors that have the same mechanism of action as ezetimibe. Additional objects will be evident from the following detailed description.

### DETAILED DESCRIPTION OF THE INVENTION

The novel cholesterol absorption inhibitors of the instant invention are compounds of structural Formula I

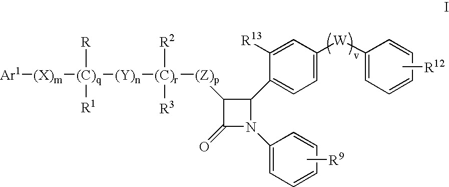

and the pharmaceutically acceptable salts thereof, wherein
Ar¹ is selected from the group consisting of aryl and R⁴-substituted aryl;
X, Y and Z are independently selected at each occurrence from the group consisting of —CH₂—, —CH(C₁₋₆alkyl)- and —C(C₁₋₆alkyl)₂-;
W is independently selected at each occurrence from the group consisting of —CH₂—, —CH(C₁₋₆alkyl)- and —C(C₁₋₆alkyl)₂-;
v is an integer selected from 0, 1, 2, 3, 4, 5 and 6;
R¹ is selected from the group consisting of —OR⁶, —O(CO)R⁶, —O(CO)OR⁸, —O(CO)NR⁶R⁷, a sugar residue, a disugar residue, a trisugar residue and a tetrasugar residue;
R¹ is selected from the group consisting of —H, —C₁₋₆alkyl and aryl, or R and R¹ together are oxo;
R² is selected from the group consisting of —OR⁶, —O(CO)R⁶, —O(CO)OR⁸ and —O(CO)NR⁶R⁷;
R³ is selected from the group consisting of —H, —C₁₋₆alkyl and aryl, or R² and R³ together are oxo;
q and r are integers each independently selected from 0 and 1 provided that at least one of q and r is 1;
m, n, and p are integers each independently selected from 0, 1, 2, 3 and 4, provided that the sum of m, n, p, q, and r is 1, 2, 3, 4, 5 or 6;
t is an integer selected from 0, 1 and 2;
R⁴ is 1-5 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀alkyl-COOR⁵, —O—C₁₋₁₀alkyl-CONR⁵R⁶, and fluoro;
R⁵, R⁶ and R⁷ are independently selected at each occurrence from the group consisting of —H, —C₁₋₆alkyl, aryl and aryl-substituted-C₁₋₆alkyl;
R⁸ is selected from the group consisting of —C₁₋₆alkyl, aryl and aryl-substituted-C₁₋₆alkyl;
R⁹ is selected from the group consisting of —C₁₋₈alkyl-Hetcy, —(CH₂)₀₋₂CH═CH—C₀₋₆alkyl-Hetcy, —C≡C—C₀₋₆alkyl-Hetcy, —C₁₋₈alkyl-NH-Hetcy, —C₁₋₈alkyl-NR¹⁰R¹¹, —(CH₂)₀₋₂CH═CH—C₁₋₆alkyl-NR¹⁰R¹¹, —C≡C—C₁₋₆alkyl-NR¹⁰R¹¹, and —C₁₋₈alkyl-SO₂R⁸;
   Hetcy is selected from the group consisting of:
(a) a 5-membered aromatic or partially unsaturated heterocyclic ring containing 1 to 4 heteroatoms selected from 1 to 4 of N, zero to 1 of S, and zero to 1 of O, wherein the heterocyclic ring is optionally mono- or di-substituted with R¹⁴,
(b) a 6-membered aromatic heterocyclic ring containing 1 to 3 N heteroatoms, wherein the heterocyclic ring is optionally mono- or di-substituted with R¹⁴, and
(c) a 6-membered saturated heterocyclic ring containing 1 to 3 heteroatoms selected from 1-3 of N, zero to 1 of O, and zero to 1 of S(O)ₜ, and wherein the heterocyclic ring is optionally mono- or di-substituted with R¹⁴;
R^{10a} is —C₁₋₃alkyl optionally substituted with one or more substituents selected form the group consisting of —OH, 1-3 of fluoro and phenyl;
R¹⁰ is independently selected at each occurrence from the group consisting of —H and —C₁₋₃alkyl optionally substituted with one or more substituents selected form the group consisting of —OH, 1-3 of fluoro and phenyl;
R¹¹ is selected from the group consisting of —H, —C(O)—C₁₋₃alkyl, —C(O)—NR¹⁰R¹⁰, —SO₂—C₁₋₃alkyl, —SO₂-phenyl and —C₁₋₃alkyl optionally substituted with one or more substituents selected form the group consisting of —OH, 1-3 of fluoro and phenyl;
R¹² is selected from the group consisting of (a) —C₁₋₁₅alkyl mono- or poly-substituted with one or more substituents selected from the group consisting of —OH and —COOH, (b) —C₂₋₁₅alkenyl mono- or poly-substituted with one or more substituents selected from the group consisting of —OH and —COOH, (c) —C₂₋₁₅alkynyl mono- or poly-substituted with one or more substituents selected from the group consisting of —OH and —COOH, (d) —C₁₋₃alkyl-C₃₋₆cycloalkyl wherein each carbon in the cycloalkyl ring is optionally substituted with —OH or —COOH, (e) a sugar residue, and (f) —C₁₋₃alkyl substituted with a sugar residue;
R¹³ is selected from the group consisting of —H, —F, and —OH; and
R¹⁴ is independently selected at each occurrence from the group consisting of:
   —R^{10a}, —C₁₋₃alkyl-COOR¹⁰, —C₁₋₃alkyl-C(O)NR¹⁰R¹⁰, —C₁₋₃alkyl-SO₂—R^{10a}, —C₁₋₃alkyl-O—R^{10a}, —COOR¹⁰, —OC(O)—R^{10a}, —C(O)NR¹⁰R¹⁰, —NR¹⁰R¹⁰, oxo, and hydroxy.

In one embodiment of this invention are compounds of Formula I wherein the sum of m, q and n is 1, 2, 3, 4, or 5 when p is 0 and r is 1.

In another embodiment of this invention are compounds of Formula I wherein r is zero and m is zero.

In another embodiment of this invention are compounds Formula I having structural Formula Ia,

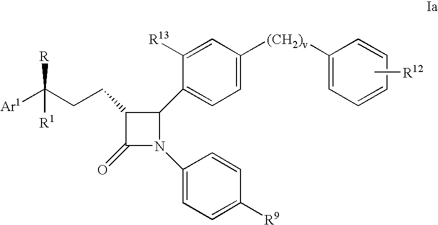

and the pharmaceutically acceptable salts thereof, wherein the variables (Ar¹, R, R¹, R⁹, R¹², R¹³, v, etc.) are as defined in Formula I.

In another embodiment of this invention are compounds Formula I and Formula Ia having structural Formula Ib,

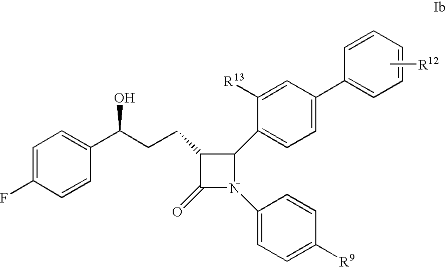

and the pharmaceutically acceptable salts thereof, wherein the variables (R⁹, R¹², R¹³, v, etc.) are as defined in Formula I.

In another embodiment of this invention are compounds of Formula I and Ia wherein v is 0 (zero). An alternative embodiment are compounds of Formula I and Ia wherein v is selected from 1, 2, 3, 4, 5 and 6, and more particularly 2, 3, 4, 5 and 6. In a class of this alternative embodiment are compounds of Formula I and Ia wherein v is selected from 1, 2, 3, 4, 5 and 6 and W is —CH₂—.

In another embodiment of this invention are compounds of Formula I and Ia wherein Ar¹ is selected from the group consisting of aryl and R⁴-substituted aryl wherein R⁴ is 1-2 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀alkyl-COOR⁵, —O—C₁₋₁₀alkyl-CONR⁵R⁶ and fluoro. In a class of this embodiment, Ar¹ is unsubstituted, mono- or di-substituted phenyl. In a sub-class, Ar¹ is phenyl mono-substituted with fluoro, and particularly 4-fluoro-phenyl.

In another embodiment of this invention are compounds of Formula I and Ia wherein R is —OR⁶; in a class of this embodiment, R is —OH.

In another embodiment of this invention are compounds of Formula I and Ia wherein R¹ is —H.

In another embodiment of this invention are compounds of Formula I wherein R² is —OR⁶; in a class of this embodiment, R² is —OH.

In another embodiment of this invention are compounds of Formula I wherein R³ is —H.

In another embodiment of this invention are compounds of Formula I, Ia and Ib wherein R⁹ is selected from the group consisting of —C₁₋₈alkyl-Hetcy, —(CH₂)₀₋₂CH═CH—C₀₋₆alkyl-Hetcy, —C≡C—C₀₋₆alkyl-Hetcy and —C₁₋₈alkyl-NH-Hetcy, and more particularly it is —C₁₋₈alkyl-Hetcy. In a class of this embodiment R⁹ is —C₁₋₈ n-alkyl-Hetcy, and more particularly it is —C₂₋₆ n-alkyl-Hetcy. In another class of this embodiment R⁹ is —CH═CH—C₀₋₆ n-alkyl-Hetcy, and more particularly it is —CH═CH—CO-4 n-alkyl-Hetcy. In another class of this embodiment R⁹ is —C≡C—CO-6 n-alkyl-Hetcy, and more particularly it is —C≡C—C₀₋₄ n-alkyl-Hetcy. In another class of this embodiment, R⁹ is —C₁₋₃alkyl-NH-Hetcy.

In another embodiment of this invention are compounds of Formula I, Ia and Ib wherein Hetcy is a 5-membered aromatic or partially unsaturated heterocyclic ring containing 1 to 4 heteroatoms selected from 1 to 4 of N, zero to 1 of S, and zero to 1 of O, wherein the heterocyclic ring is optionally mono- or di-substituted with R¹⁴. Examples of such heterocyclic rings within the meaning of Hetcy include but are not limited to the following, each of which may be optionally mono- or di-substituted with R¹⁴:

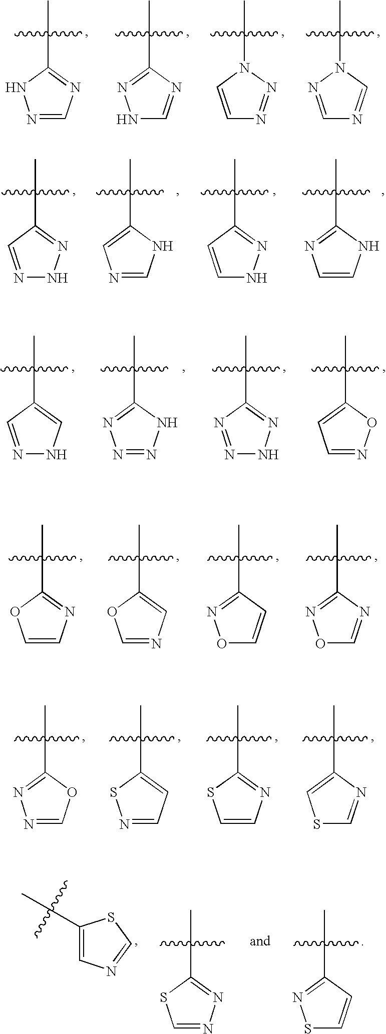

In another embodiment of this invention are compounds of Formula I, Ia and Ib wherein Hetcy is a 6-membered aromatic heterocyclic ring containing 1 to 3 N heteroatoms, and particularly wherein the ring contains 1-2 of N, wherein the heterocyclic ring is optionally mono- or di-substituted with R¹⁴. Examples of such heterocyclic rings within the meaning of Hetcy include but are not limited to the following, each of which may be optionally mono- or di-substituted with R¹⁴:

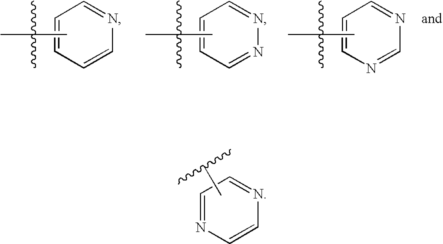

In another embodiment of this invention are compounds of Formula I, Ia and Ib wherein Hetcy is a 6-membered saturated heterocyclic ring containing 1 to 3 heteroatoms selected from 1-3 of N, zero to 1 of O, and zero to 1 of S(O)ₜ, wherein the heterocyclic ring is optionally substituted with R¹⁴. Examples of such heterocyclic rings within the meaning of Hetcy include but are not limited to the following, each of which may be optionally mono- or di-substituted with R¹⁴:

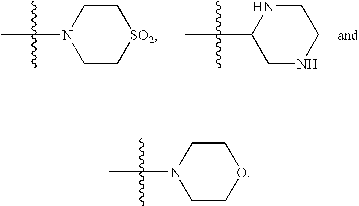

In another embodiment of this invention are compounds of Formula I, Ia and Ib wherein R⁹ is —C₁₋₈alkyl-NR¹⁰R¹¹. In a class of this embodiment, R¹¹ is selected from —SO₂—C₁₋₃alkyl and —SO₂-phenyl. In a sub-class of this class, R⁹ is —C₁₋₈alkyl-NR¹⁰—SO₂CH₃, and more particularly it is —C₃₋₆alkyl-NR¹⁰—SO₂CH₃.

In another embodiment of this invention are compounds of Formula I, Ia and Ib wherein R⁹ is —(CH₂)₀₋₂CH═CH—C₁₋₆alkyl-NR¹⁰R¹¹. In a class of this embodiment, R¹¹ is selected from —SO₂—C₁₋₃alkyl and —SO₂-phenyl. In a sub-class of this class, R⁹ is —C═C—C₁₋₆alkyl-NR¹⁰—SO₂CH₃, and more particularly it is —CH═CH—C₁₋₄-alkyl-NR¹⁰—SO₂CH₃.

In another embodiment of this invention are compounds of Formula I, Ia and Ib wherein R⁹ is —C≡C—C₁₋₆alkyl-NR¹⁰R¹¹. In a class of this embodiment, R¹¹ is selected from —SO₂—C₁₋₃alkyl and —SO₂-phenyl. In a sub-class of this class, R⁹ is —C≡C—C₁₋₆alkyl-NR¹⁰—SO₂CH₃, and more particularly it is —C≡C—C₁₋₄alkyl-NR¹⁰—SO₂CH₃.

In another embodiment of this invention are compounds of Formula I, Ia or Ib wherein R⁹ is —C₁₋₈alkyl-SO₂—R⁸. In a class of this embodiment, R⁸ is —C₁₋₆alkyl, more particularly it is —C₁₋₃alkyl, and most particularly it is methyl.

In another embodiment of this invention are compounds of Formula I, Ia or Ib wherein R¹⁴ is selected from —C(O)NH₂, —COOH, methyl, and oxo.

In another embodiment of this invention are compounds of Formula I, Ia or Ib wherein R¹⁰ is selected from —H and methyl.

In another embodiment of this invention are compounds of Formula I, Ia or Ib wherein R¹¹ is selected from —SO₂—C₁₋₃alkyl and —SO₂-phenyl, and more particularly R¹¹ is —SO₂—CH₃.

In another embodiment of this invention are compounds of Formula I, Ia and Ib wherein R¹² is —C₁₋₁₅alkyl mono- or poly-substituted with one or more substituents selected from the group consisting of —OH and —COOH. In a class of this embodiment, R¹² is —C₁₋₈alkyl mono- or poly-substituted with one or more substituents selected from the group consisting of —OH and —COOH. In a sub-class of this class, R¹² is —C₃₋₆ alkyl mono- or poly-substituted with one or more substituents selected from the group consisting of —OH and —COOH. In a further sub-class of this class, R¹² is —(CH₂)₂₋₃—C(OH)(CH₂OH)₂.

In another embodiment of this invention are compounds of Formula I, Ia or Ib wherein R¹² is —C₂₋₁₅alkenyl mono- or poly-substituted with one or more substituents selected from the group consisting of —OH and —COOH. In a class of this embodiment, R¹² is —C₂₋₈alkenyl mono- or poly-substituted with one or more substituents selected from the group consisting of —OH and —COOH. In a sub-class of this class, R¹² is —C₃₋₆ alkenyl mono- or poly-substituted with one or more substituents selected from the group consisting of —OH and —COOH. In a further sub-class of this class, R¹² is —(CH₂)₀₋₁—CH═CH—C(OH)(CH₂OH)₂.

In another embodiment of this invention are compounds of Formula I, Ia or Ib wherein R¹² is —C₂₋₁₅alkynyl mono- or poly-substituted with one or more substituents selected from the group consisting of —OH and —COOH. In a class of this embodiment, R¹² is —C₂₋₈alkynyl mono- or poly-substituted with one or more substituents selected from the group consisting of —OH and —COOH. In a sub-class of this class, R¹² is —C₃₋₆ alkynyl mono- or poly-substituted with one or more substituents selected from the group consisting of —OH and —COOH. In a further sub-class of this class, R¹² is —(CH₂)₀₋₁—C≡C—C(OH)(CH₂OH)₂.

In another embodiment of this invention are compounds of Formula I, Ia and Ib wherein R¹² is a sugar residue. Examples of such sugar residues within the meaning of R¹² include but are not limited to the following:

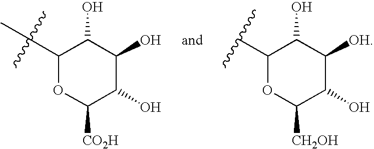

Each embodiment, class or sub-class described above for each variable (i.e., Ar¹, R, R¹, R⁹, R¹², etc.) in Formulas I, Ia and Ib may be combined with one or more of the embodiments, classes or sub-classes described above for one or more other variables, and all such sub-generic combinations are included within the scope of this invention.

As used herein “alkyl” is intended to include both branched- and straight-chain saturated aliphatic hydrocarbon groups having the specified number of carbon atoms. Examples of alkyl groups include, but are not limited to, methyl (Me), ethyl (Et), n-propyl (Pr), n-butyl (Bu), n-pentyl, n-hexyl, and the isomers thereof such as isopropyl (i-Pr), isobutyl (i-Bu), secbutyl (s-Bu), tertbutyl (t-Bu), 1-methylpropyl, 2-methylbutyl, 3-methylbutyl, isopentyl, isohexyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means a monocyclic saturated carbocyclic ring. Examples of cycloalkyl include, but are not limited to, cyclopropyl, cyclobutyl, cyclopentyl, cyclohexyl, and the like.

Certain alkyl groups defined herein may be “mono- or poly-substituted with one or more substituents selected from the group consisting of —OH and —COOH,” meaning that one or more hydroxyl or carboxyl substituents is present on the alkyl group, and that each carbon atom available for substitution in the alkyl group may independently be unsubstituted or mono-substituted with hydroxyl or carboxyl provided that at least one carbon atom is substituted with hydroxyl or carboxyl. This encompasses alkyl groups where every available carbon atom is mono-substituted with hydroxyl or carboxyl as well as those where fewer than all available carbon atoms are mono-substituted with hydroxyl or carboxyl. This also encompasses alkyl groups where only hydroxyl substitutions occur, alkyl groups where only carboxyl substitutions occur, and alkyl groups where a combination of hydroxyl and carboxyl substitutions occur.

As used herein, “aryl” is intended to include phenyl (Ph), naphthyl, indenyl, tetrahydronaphthyl or indanyl. Phenyl is preferred.

Hydroxyl protecting groups may be used on intermediates during the synthetic procedures for making final products within the scope of this invention. Suitable protecting groups (designated as “PG” herein) for the hydroxyl groups, for example those in R¹² and R¹³, include but are not limited to those that are known to be useful as hydroxyl protecting groups, such as for example benzyl, acetyl, benzoyl, tert-butyldiphenylsilyl, trimethylsilyl, para-methoxybenzyl, benzylidine, dimethylacetal and methoxy methyl. Conditions required to selectively add and remove such protecting groups are found in standard textbooks such as Greene, T, and Wuts, P. G. M., Protective Groups in Organic Synthesis, John Wiley & Sons, Inc., New York, N.Y., 1999.

The terms “heterocycle” and derivatives thereof such as “heterocyclyl” and “heterocyclic ring” mean an aromatic, partially unsaturated or saturated ring containing one or more carbon atoms and one or more heteroatoms such as nitrogen, oxygen and sulfur, but may be more specifically defined where appropriate in the specification, for example with respect to degree of saturation, number of members (i.e. atoms) in the ring and/or the type and quantity of heteroatoms in the ring. The point of attachment in a compound structure may be via any carbon or nitrogen in the heterocyclic ring which results in the creation of a stable structure, unless specified otherwise. The heterocyclic ring may be substituted on any available carbon or nitrogen in the ring which results in the creation of a stable structure, unless specified otherwise.

Compounds of Formula I may contain one or more asymmetric centers and can thus occur as racemates and racemic mixtures, single enantiomers, enantiomeric mixtures, diastereomeric mixtures and individual diastereomers. All such isomeric forms of the compounds of Formula I are included within the scope of this invention. Furthermore, some of the crystalline forms for compounds of the present invention may exist as polymorphs and as such are intended to be included in the present invention. In addition, some of the compounds of the instant invention may form solvates with water or organic solvents. Such hydrates and solvates are also encompassed within the scope of this invention. Some of the compounds described herein may contain olefinic double bonds, and unless specified otherwise, are meant to include both E and Z geometric isomers, singly or as a mixture.

Some of the compounds encompassed herein may exist as tautomers, e.g., keto-enol tautomers. For the purpose of illustration, when Hetcy is a 5-membered heterocyclic ring substituted with oxo, the resulting compound may be capable of tautomerism, as exemplified below:

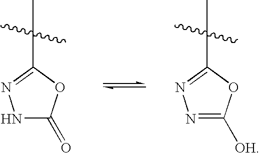

Due to their activity as cholesterol absorption inhibitors, the compounds of the present invention can be used in screening assays, where the assay is designed to identify new cholesterol absorption inhibitors. Radioactive isotopes of the compounds of Formula I are particularly useful in such assays, for example compounds of Formula I wherein sulfur is replaced with “hot”—³⁵S—, and particularly wherein the radioactive sulfur isotope is incorporated within the R⁹ moiety. All such radioactive isotopes of the compounds of Formula I are included within the scope of this invention.

Reference to the compounds of this invention as those of “Formula I,” “Formula Ia,” and “Formula Ib” is intended herein to encompass compounds falling within the scope of each of these structural formulas including pharmaceutically acceptable salts thereof where such salts are possible. Herein, the term “pharmaceutically acceptable salts” means non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)aminomethane.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Also, in the case of a carboxylic acid (—COOH) or alcohol group being present in the compounds of this invention, pharmaceutically acceptable esters of carboxylic acid derivatives, such as —C1-4 alkyl, —C1-4 alkyl substituted with phenyl, acetylamino and pivaloyloxymethyl, or acyl derivatives of alcohols such as O-acetyl, O-pivaloyl, O-benzoyl, O-dimethylamino and O-acetylamino, can be employed. Included within the scope of this invention are those esters and acyl groups known in the art for modifying the solubility or hydrolysis characteristics of a compound for use as sustained-release or prodrug formulations.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by inhibition of cholesterol absorption.

The term “therapeutically effective amount” is intended to mean that amount of a pharmaceutical drug that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage a patient receives can be selected so as to achieve the amount of LDL cholesterol lowering desired; the dosage a patient receives may also be titrated over time in order to reach a target LDL level. The dosage regimen utilizing a compound of the instant invention is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition.

The compounds of the instant invention are cholesterol absorption inhibitors and are useful for reducing plasma cholesterol levels, particularly reducing plasma LDL cholesterol levels, when used either alone or in combination with another active agent, such as an anti-atherosclerotic agent, and more particularly a cholesterol biosynthesis inhibitor, for example an HMG-CoA reductase inhibitor. Thus the instant invention provides methods for inhibiting cholesterol absorption and for treating lipid disorders including hypercholesterolemia, comprising administering a therapeutically effective amount of a compound of Formula I to a person in need of such treatment. The term hypercholesterolemia includes but is not limited to homozygous familial hypercholesterolemia (HoFH) and heterozygous familial hypercholesterolemia (HeFH) and therefore the compounds of Formula I can be used treat HoHF and HeHF patients. These compounds can also be used for the treatment of mixed hyperlipidemia which is characterized by an elevated LDL cholesterol level and elevated triglycerides level along with an undesirably low HDL cholesterol level. Compounds of Formula I can also be used to treat or prevent sitosterolemia and/or to lower the concentration of one or more sterols other than cholesterol in the plasma or tissue of a patient.

Further provided are methods for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a mammal who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

A compound of Formula I may be administered to prevent or reduce the risk of occurrence, or recurrence where the potential exists, of a coronary heart disease event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of Formula I to a patient at risk for such an event. The patient may or may not have atherosclerotic disease at the time of administration, or may be at risk for developing it.

Persons to be treated with the instant therapy include those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein (HDL) cholesterol, and a family history of atherosclerotic cardiovascular disease. Published guidelines for determining those who are at risk of developing atherosclerotic disease can be found in: Executive Summary of the Third Report of the National Cholesterol Education Program (NCEP) Expert Panel on Detection, Evaluation, and Treatment of High Blood Cholesterol in Adults (Adult Treatment Panel III), JAMA, 2001; 285 pp. 2486-2497. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The oral dosage amount of the compound of Formula I is from about 0.1 to about 30 mg/kg of body weight per day, preferably about 0.1 to about 15 mg/kg of body weight per day. For an average body weight of 70 kg, the dosage level is therefore from about 5 mg to about 1000 mg of drug per day. However, dosage amounts will vary depending on factors as noted above, including the potency of the particular compound. Although the active drug of the present invention may be administered in divided doses, for example from two to four times daily, a single daily dose of the active drug is preferred. As examples, the daily dosage amount may be selected from, but not limited to, 5 mg, 10 mg, 15 mg, 20 mg, 25 mg, 40 mg, 50 mg, 75 mg, 80 mg, 100 mg and 200 mg.

The active drug employed in the instant therapy can be administered in such oral forms as tablets, capsules, pills, powders, granules, elixirs, tinctures, suspensions, syrups, and emulsions. Oral formulations are preferred, and particularly solid oral formulations such as tablets.

For compounds of Formula I, administration of the active drug can be via any pharmaceutically acceptable route and in any pharmaceutically acceptable dosage form. This includes the use of oral conventional rapid-release, time controlled-release and delayed-release (such enteric coated) pharmaceutical dosage forms. Additional suitable pharmaceutical compositions for use with the present invention are known to those of ordinary skill in the pharmaceutical arts; for example, see Remington's Pharmaceutical Sciences, Mack Publishing Co., Easton, Pa.

In the methods of the present invention, the active drug is typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants, for example butylated hydroxyanisole (BHA), 2,6-di-tert-butyl-4-methylphenol (BHT), propyl gallate, sodium ascorbate, citric acid, calcium metabisulphite, hydroquinone, and 7-hydroxycoumarin, particularly BHA, propyl gallate and combinations thereof, can also be added to stabilize the dosage forms. When a compound of Formula I is formulated together with an HMG-CoA reductase inhibitor such as simvastatin, the use of at least one stabilizing agent is preferred in the composition. Other suitable components include gelatin, sweeteners, natural and synthetic gums such as acacia, tragacanth or alginates, carboxymethylcellulose, polyethylene glycol, waxes and the like.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

One or more additional active agents may be administered in combination with a compound of Formula I, and therefore an embodiment of the instant invention encompasses a drug combination. The drug combination encompasses a single dosage formulation comprised of the compound of Formula I and additional active agent or agents, as well as administration of each of the compound of Formula I and the additional active agent or agents in separate dosage formulations, which allows for concurrent or sequential administration of the active agents. The additional active agent or agents can be lipid modifying agents, particularly a cholesterol biosynthesis inhibitor such as an HMG-CoA reductase inhibitor, or agents having other pharmaceutical activities, or agents that have both lipid-modifying effects and other pharmaceutical activities. Examples of HMG-CoA reductase inhibitors useful for this purpose include statins in their lactonized or dihydroxy open acid forms and pharmaceutically acceptable salts and esters thereof, including but not limited to lovastatin (MEVACOR®; see U.S. Pat. No. 4,342,767); simvastatin (ZOCOR®; see U.S. Pat. No. 4,444,784); dihydroxy open-acid simvastatin, particularly the ammonium or calcium salts thereof; pravastatin, particularly the sodium salt thereof (PRAVACOL®; see U.S. Pat. No. 4,346,227); fluvastatin particularly the sodium salt thereof (LESCOL®; see U.S. Pat. No. 5,354,772); atorvastatin, particularly the calcium salt thereof (LIPITOR®; see U.S. Pat. No. 5,273,995); rosuvastatin (CRESTOR®; see U.S. Pat. No. 5,260,440); and pitavastatin also referred to as NK-104 (see PCT international publication number WO 97/23200). Examples of additional active agents which may be employed include but are not limited to one or more of FLAP inhibitors; 5-lipoxygenase inhibitors; additional cholesterol absorption inhibitors such as ezetimibe (ZETIA®), described in U.S. Pat. Nos. Re. 37721 and 5,846,966; cholesterol ester transfer protein (CETP) inhibitors, for example JTT-705, also known as CP529,414; HMG-CoA synthase inhibitors; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT1 and -2; microsomal triglyceride transfer protein (MTP) inhibitors; niacin; niacin receptor agonists such as acipimox and acifran, as well as niacin receptor partial agonists; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidinediones as well as those PPARγ agonists outside the thiazolidinedione structural class; PPAR α agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABC1 gene expression; FXR ligands including both inhibitors and agonists; and LXR ligands including both inhibitors and agonists of all sub-types of this receptor, e.g. LXRα and LXRβ; bisphosphonate compounds such as alendronate sodium; and cyclooxygenase-2 inhibitors such as celecoxib and etoricoxib.

A therapeutically or prophylactically effective amount, as appropriate, of a compound of Formula I can be used for the preparation of a medicament useful for inhibiting cholesterol absorption, as well as for treating and/or reducing the risk for diseases and conditions affected by inhibition of cholesterol absorption, such as treating lipid disorders, preventing or reducing the risk of developing atherosclerotic disease, halting or slowing the progression of atherosclerotic disease once it has become clinically manifest, and preventing or reducing the risk of a first or subsequent occurrence of an atherosclerotic disease event. For example, the medicament may be comprised of about 5 mg to about 1000 mg of a compound of Formula I. The medicament comprised of a compound of Formula I may also be prepared with one or more additional active agents, such as those described supra.

The following Rat and/or Mouse assay can be used to test compounds for their ability to inhibit cholesterol absorption.

Cholesterol Absorption Assay in Rat: CD male rats (n=5/group), aged 5 weeks, were dosed orally with 0.5 ml 0.25% methyl cellulose solution with or without test compound or ezetimibe (0.0003-1 mg/kg). 0.5 to 16 hrs later all of the rats were dosed orally with 0.5 ml INTRALIPID™ containing 5 μCi [³H]-cholesterol per rat. Five hours later, the animals were euthanized, and liver and blood were collected. Cholesterol counts in liver and plasma were determined, and percent inhibition of cholesterol absorption was calculated.

Cholesterol Absorption Assay in Mice: C57BL/6 male mice (n=6/group), aged 10-14 weeks, were dosed orally with 0.2 ml 0.25% methyl cellulose solution with or without test compound or ezetimibe (0.12-10 mg/kg). Thirty minutes later all of the mice were dosed orally with 0.2 ml INTRALIPID™ containing 2 μCi [³H]-cholesterol per mouse. Five hours later, the animals were euthanized, and liver and blood were collected. Cholesterol counts in liver and plasma were determined, and percent inhibition of cholesterol absorption was calculated.

The compounds of structural Formula I of the present invention can be prepared according to the procedures of the following Scheme and Examples, using appropriate materials, and are further exemplified by specific examples which follow. Moreover, by utilizing the procedures described herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds.

A variety of chromatographic techniques may be employed in the preparation of the compounds. These techniques include, but are not limited to: High Performance Liquid Chromatography (HPLC) including normal-, reversed- and chiral-phase; Medium Pressure Liquid Chromatography (MPLC); Super Critical Fluid Chromatography; preparative Thin Layer Chromatography (prep TLC); flash chromatography with silica gel or reversed-phase silica gel; ion-exchange chromatography; and radial chromatography. All temperatures are degrees Celsius unless otherwise noted.

Some abbreviations that may be used herein include:

Ac Acyl (CH₃C(O)—)

Aq. Aqueous

Bn Benzyl

C. Celsius

calc. Calculated

DCM Dichloromethane

DEAD Diethylazodicarboxylate

DIAD Diisopropylazodicarboxylate

DIEA N,N-diisopropylethylamine

DMAP 4-dimethylaminopyridine

DMF N,N-dimethylformamide

equiv. Equivalent(s)

ES-MS Electron Spray Ion-Mass Spectroscopy

Et₂O Ethyl ether

EtOAc Ethyl acetate

EtOH Ethanol

h Hour(s)

MeOH Methanol

min Minute(s)

mp Melting point

MS Mass spectrum

Prep. Preparative

r.t. (or rt) Room temperature

sat. Saturated

TBAF Tetrabutylammonium fluoride

TBAI tetrabutylammonium iodide

TBS Tert-butyl dimethylsilyl

TEA Triethyl amine

Tf trifluoromethylsulfonyl

TFA Trifluoroacetic acid

THF Tetrahydrofuran

TMS trimethylsilyl

The general Schemes below illustrate a method for the syntheses of compounds of the present invention. All substituents and variables (e.g., R¹, R², Ar¹, X, Y, etc.) are as defined above in Formula I unless indicated otherwise. Examples of hydroxyl protecting groups (PG) include, for example, benzyl, acetate, acetal or any other suitable oxygen protecting group, or combinations thereof, compatible with earlier or subsequent chemical reactions. As an example, R^{12a} includes but is not limited to —C₁₋₆alkyl-OBn,

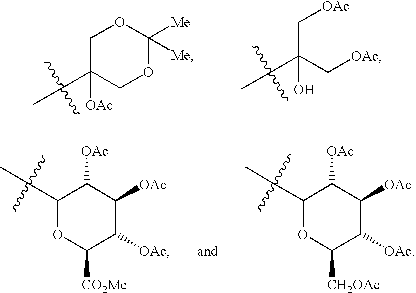

The following definitions are used in the schemes below:

R^{9a}=—C₁₋₆alkyl-Hetcy, —C₁₋₆alkyl-NH-Hetcy, —C₁₋₆alkyl-SO₂R⁸, or —C₁₋₆alkyl-NR¹⁰R¹¹;
R^{12a}=R¹², hydroxy-protected R¹²;
R^{12b}=—C₁₋₁₃alkyl mono- or poly-substituted with —OH or —OPG and/or —COOH, wherein the hydroxyl protecting groups can be removed to form the final product;
and R^{13a}=R¹³ and —OPG such as OBn, OAllyl, OPh.

In Scheme I, the intermediate I-1 may be reacted with a substituted phenyl halide (I-2) in the presences of a suitable palladium catalyst such as palladium tetrakis and a base such as triethylamine or potassium carbonate in a solvent system, such as toluene and ethanol, to afford a compound containing a substituted biphenyl moiety as the type represented by I-3. Intermediate I-3 may then be converted to I-4 by treatment with guanidine and triethylamine in methanol to selectively remove the phenolic acetate; then converting the intermediate phenol to the triflate via treatment with bis(trifluoromethylsulfonyl)amino pyridine in the presence of either triethylamine or N,N diisopropyl-N-ethyl amine in dichloromethane medium. Intermediate I-4 is then treated with a terminal alkyne of type I-5 containing the R^{9a} group in the presence of a suitable palladium catalyst such as tetrakistriphenylphosphine palladium(0) or [1,1′-bis(diphenylphosphino)ferrocene]dichloropalladium(II) or the like, and copper(I) iodide and an initiator such as tetra-n-butylammonium iodide. The reaction is usually performed in an inert organic solvent such as DMF, between room temperature and 100° C., for a period of 6-48 h, and the product is an internal alkyne of structural formula I-6. Hydrolysis or cleavage of any hydroxyl protecting groups may be performed at this time, or non-benzylic protecting groups can be removed prior to the hydrogenation step. For example, diols protected as acetals that are contained in R^{12a} may be removed by treatment with aqueous acid. When R^{12a} contains one or more acetate groups, deprotection with potassium cyanide or potassium trimethylsilanoate in an alcohol solvent such as ethanol at ambient temperature or heated to 50° C. for I-2 hours affords the free hydroxyl groups to form compounds of the present invention I-7. An additional deprotection step may be included if there are useful protecting groups on the heteroaryl group know to those skilled in the art necessary to allow the chemistry to proceed in a facile fashion. These protecting groups may include trityl groups, t-butylcarbamate groups or other groups suitable for the protection of heterocyclic compounds or the functional groups attached to the heterocyclic group known to those skilled in the art. Hydrogenation of this alkyne intermediate I-7 by treatment with 10% palladium on carbon catalyst under hydrogen atmosphere in a solvent such as ethanol over 15-24 hours may achieve hydrogenation of the triple bonds along with the removal of any benzyl protecting groups in I-8. When R¹³ is the 2-benzyloxy substituent, these conditions are suitable to afford the 2-hydroxy substituted phenyl as in the structure of type I-8.

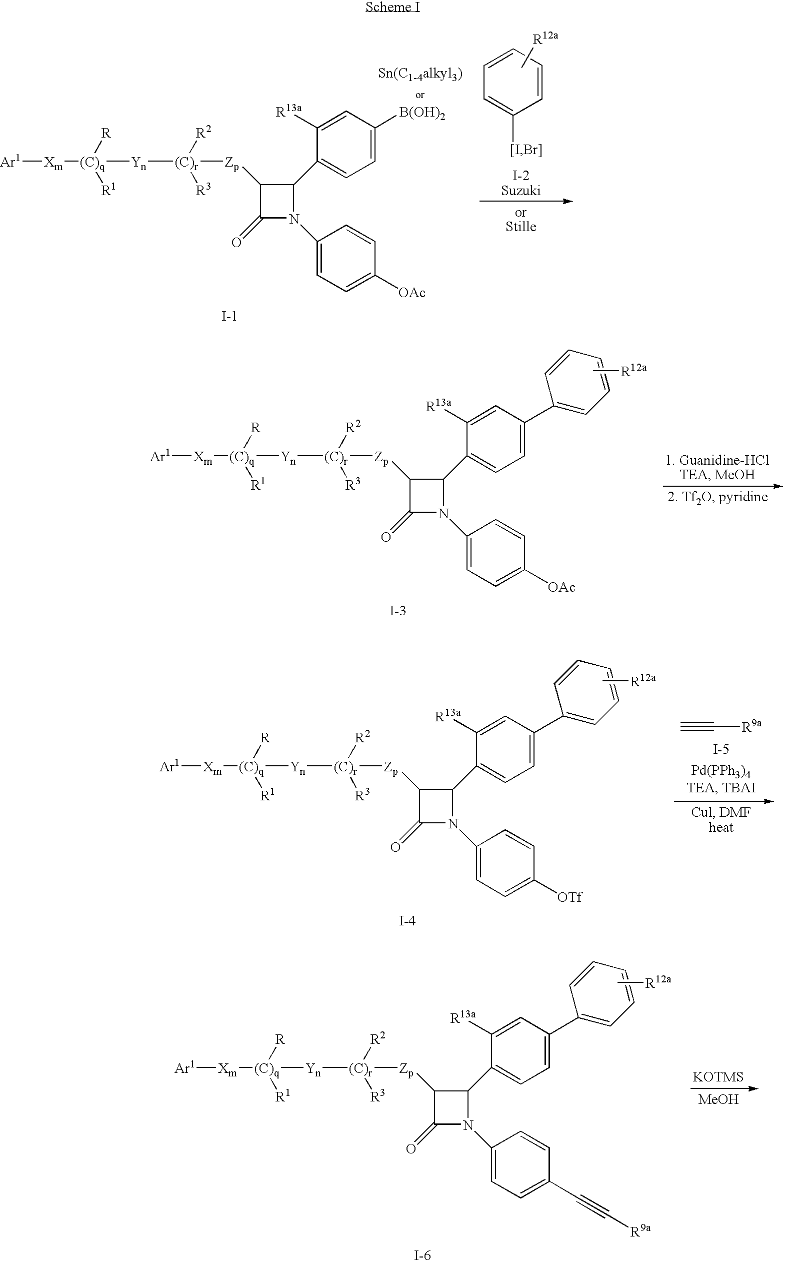

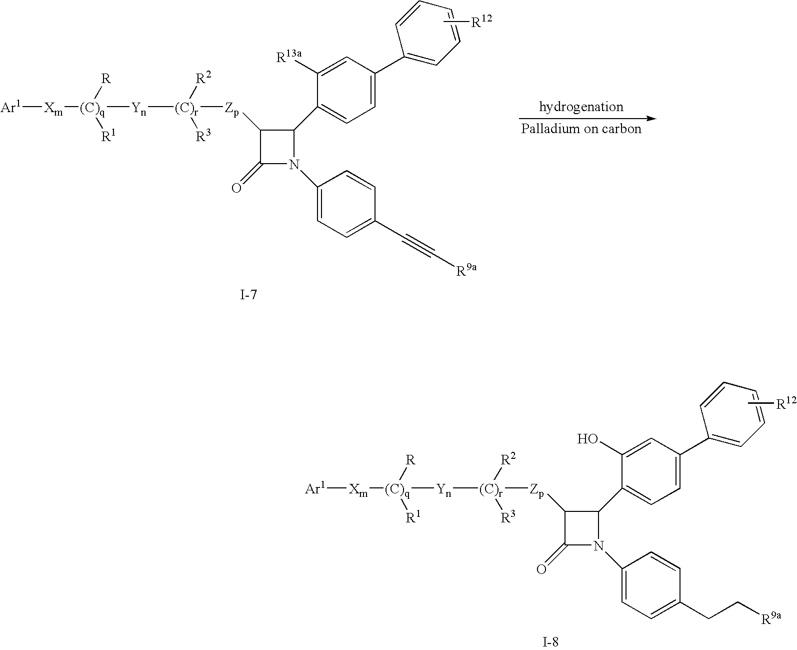

A similar synthesis route to compounds of the present invention is outlined in Scheme Ia. The triflate intermediate I-4 described in the prior scheme may undergo alkyne cross coupling with TMS-acetylene, silicon removal and then a second cross-coupling with heteroaryl halides to arrive at intermediate I-9. Hydrogenation of this alkyne intermediate I-9 by treatment with 10% palladium on carbon catalyst under hydrogen atmosphere in a solvent such as ethyl acetate over 15-24 hours may achieve hydrogenation of the triple bonds along with the removal of any benzyl protecting groups, except for substituent R^{13a} in which the benzyl protection survives these hydrogenation conditions. An additional deprotection step may be included if there are useful protecting groups on the heteroaryl group know to those skilled in the art necessary to allow the chemistry to proceed in a facile fashion. These protecting groups may include trityl groups, t-butylcarbamate groups or other groups suitable for the protection of heterocyclic compounds or the functional groups attached to the heterocyclic group known to those skilled in the art. Hydrolysis or cleavage of any remaining hydroxyl protecting groups may be performed at this time, or non-benzylic protecting groups can be removed prior to the hydrogenation step. For example, diols protected as acetals that are contained in R^{12a} may be removed by treatment with aqueous acid. When R^{12a} contains one or more acetate groups, deprotection with potassium cyanide or potassium trimethylsilanoate in an alcohol solvent such as ethanol at ambient temperature or heated to 50° C. for 1-2 hours affords the free hydroxyl groups to form compounds of the present invention I-10. When R^{13a} is present as a hydroxy-protected group, a second deprotection step using 10% palladium on carbon in ethanol under hydrogen atmosphere is required as a final deprotection to afford the 2-hydroxy substituted phenyl as in the structure of type I-11.

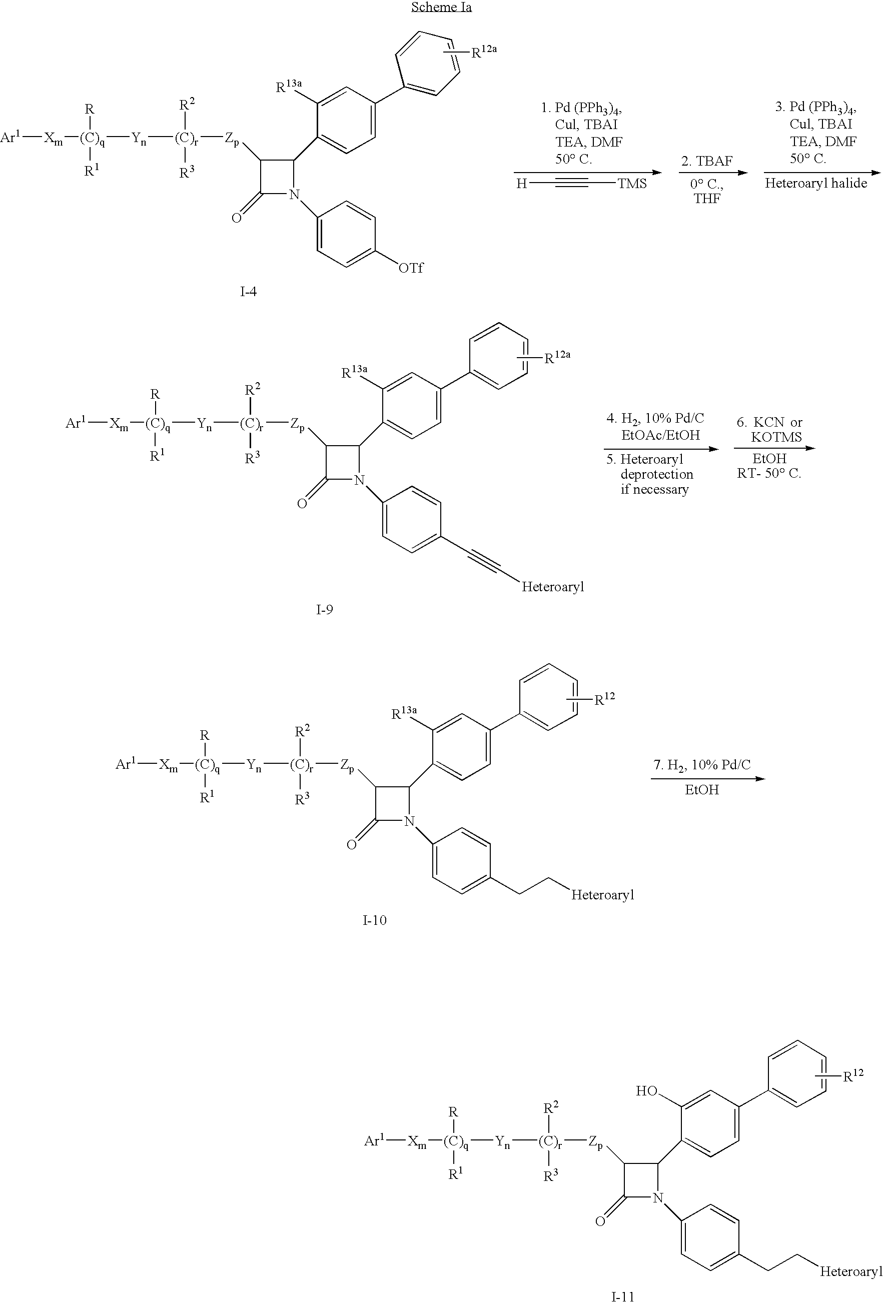

Variation in chain length between the two phenyl moieties is outlined in Scheme II. Cross-coupling of bromide intermediate I-1a with allyl or vinyl stannane intermediates may be performed in the presence of a palladium catalysts such as Pd(PPh₃)₄ or PdCl₂(PPh₃)₂ in an inert solvent such as DMF at RT or elevated temperature. The subsequent vinyl compound I-12 may be reacted in an olefin cross metathesis with a vinyl intermediate I-13 containing R^{12a} using an appropriate catalyst useful olefin metathesis known to those skilled in the art. These catalysts may include the “Shrock” catalyst or the “Zhan” catalyst to produce the intermediates of general structure I-14. The resulting triflate I-14 is treated with an alkynyl-(CH₂)ₙ-heterocyclo group of type I-5 in the presence of a suitable palladium catalyst such as tetrakistriphenylphosphine palladium(0) and copper(I) iodide with an initiator such as tetrabutylammonium iodide. The reaction is usually performed in an inert organic solvent such as DMF, at RT to 50° C., for a period of 1 to 5 hrs, yielding intermediate structure I-15. Hydrogenation of this alkenyl, alkyne intermediate I-15 by treatment with 10% palladium on carbon catalyst under hydrogen atmosphere in a solvent such as ethyl acetate over 15-24 hours may achieve hydrogenation of the triple bonds along with the removal of any benzyl protecting groups, except for substituent R^{13a} in which the benzyl protection survives these hydrogenation conditions. An additional deprotection step may be included if there are useful protecting groups on the heterocyclo group know to those skilled in the art necessary to allow the chemistry to proceed in a facile fashion. These protecting groups may include trityl groups, t-butylcarbamate groups or other groups suitable for the protection of heterocyclic compounds or the functional groups attached to the heterocyclic group known to those skilled in the art. Hydrolysis or cleavage of any remaining hydroxyl protecting groups may be performed at this time, or non-benzylic protecting groups can be removed prior to the hydrogenation step. For example, diols protected as acetals that are contained in R^{12a} may be removed by treatment with aqueous acid. When R^{12a} contains one or more acetate groups, deprotection with potassium cyanide or potassium trimethylsilanoate in an alcohol solvent such as ethanol at ambient temperature or heated to 50° C. for 1-2 hours affords the free hydroxyl groups to form compounds of the present invention I-16. When R^{13a} is present as a hydroxy-protected group, a second deprotection step using 10% palladium on carbon in ethanol under hydrogen atmosphere is required as a final deprotection to afford the 2-hydroxy substituted phenyl as in the structure of type I-17.

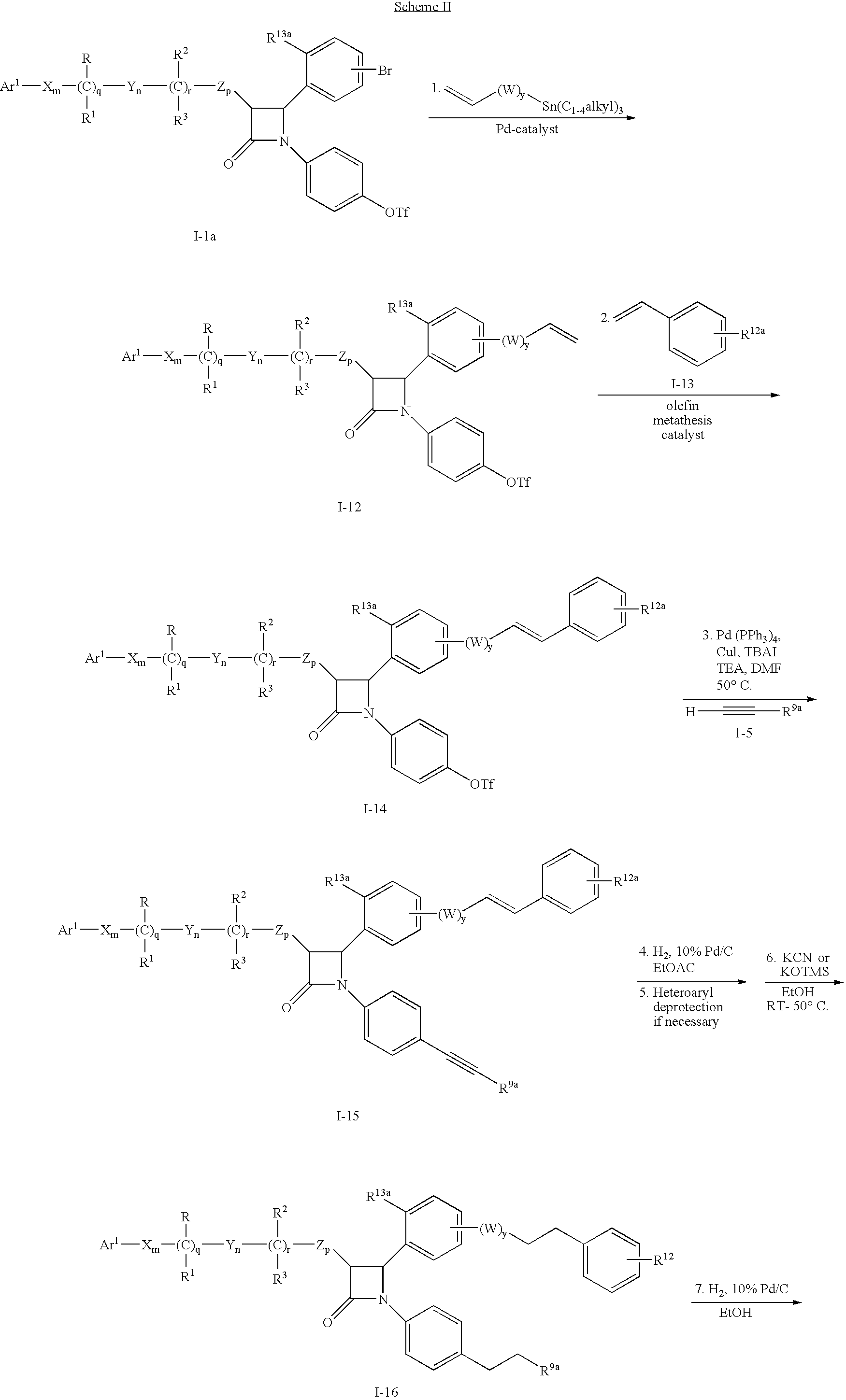

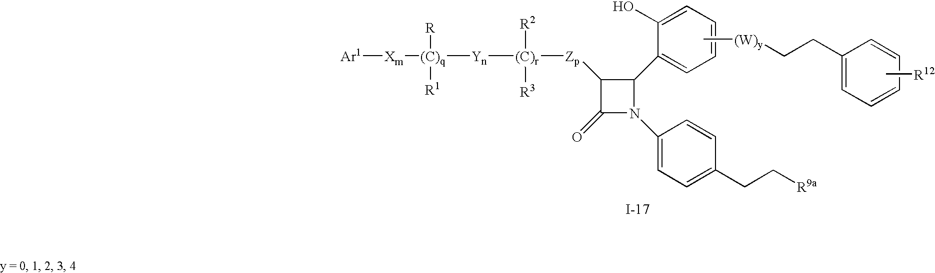

A fourth synthesis route to compounds of the present invention is outlined in Scheme IIa. The triflate intermediate I-14 described in the prior scheme may undergo alkyne cross coupling with TMS-acetylene, silicon removal and then a second cross-coupling with heteroaryl halides to arrive at intermediate I-18. The intermediate I-18 may then be converted to compounds of the present invention I-19 and I-20 by following the previously described hydrogenation and subsequent deprotection steps, as in Scheme II, necessary to complete the synthesis.

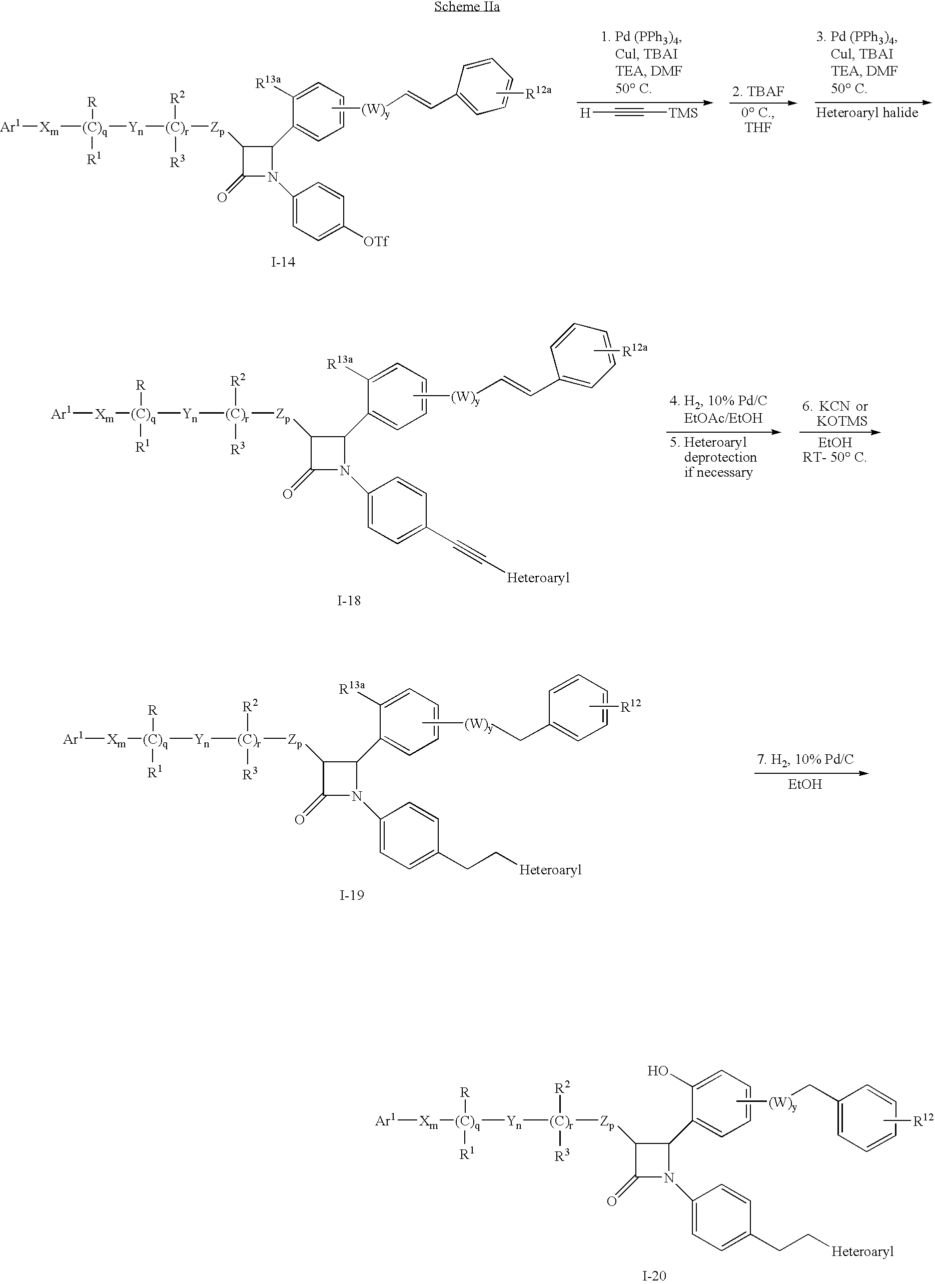

In Scheme III, the intermediate I-1 may be reacted with 1,4-, 1,3-, or 1,2-dibromobenzene or a 2, 3, or 4-bromobenzene halide in the presences of a suitable palladium catalyst such as palladium tetrakis and a base such as triethylamine or potassium carbonate in a solvent system, such as toluene and ethanol, to afford a compound containing a substituted biphenyl moiety as the type represented by I-21. Intermediate I-21 may then be treated with a terminal alkyne of type I-22 containing the R^{12a} group in the presence of a suitable palladium catalyst such as tetrakistriphenylphosphine palladium(0) or [1,1′-bis(diphenylphosphino)ferrocene]dichloropalladium(II) or the like, and copper(I) iodide and an initiator such as tetra-n-butylammonium iodide. The reaction is usually performed in an inert organic solvent such as DMF, between room temperature and 100° C., for a period of 6-48 h, and the product is an internal alkyne of structural formula I-23. Intermediate I-23 may then be converted to I-24 as previously described in Scheme I. Intermediate I-24 is then treated with a terminal alkyne of type I-5 containing the R^{9a} group in the presence of a suitable palladium catalyst such as tetrakistriphenylphosphine palladium(0) or [1,1′-bis(diphenylphosphino)ferrocene]dichloropalladium(II) or the like, and copper(I) iodide and an initiator such as tetra-n-butylammonium iodide. The reaction is usually performed in an inert organic solvent such as DMF, between room temperature and 100° C., for a period of 6-48 h, and the product is an internal di-alkyne of structural formula I-25. The intermediate I-25 may then be converted to compounds of the present invention I-26 and I-27 by following the previously described hydrogenation and subsequent deprotection steps, as in Scheme II, necessary to complete the synthesis.

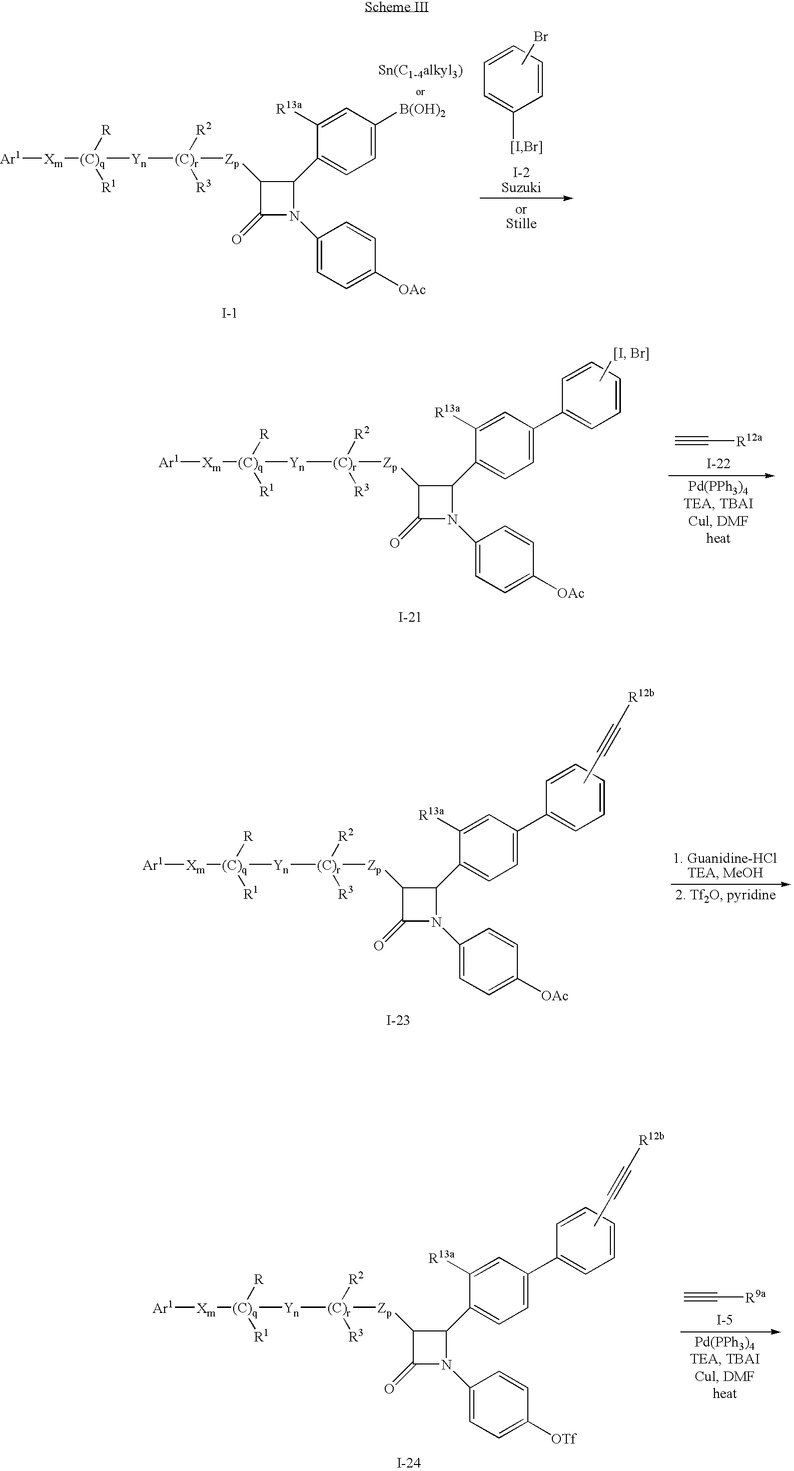

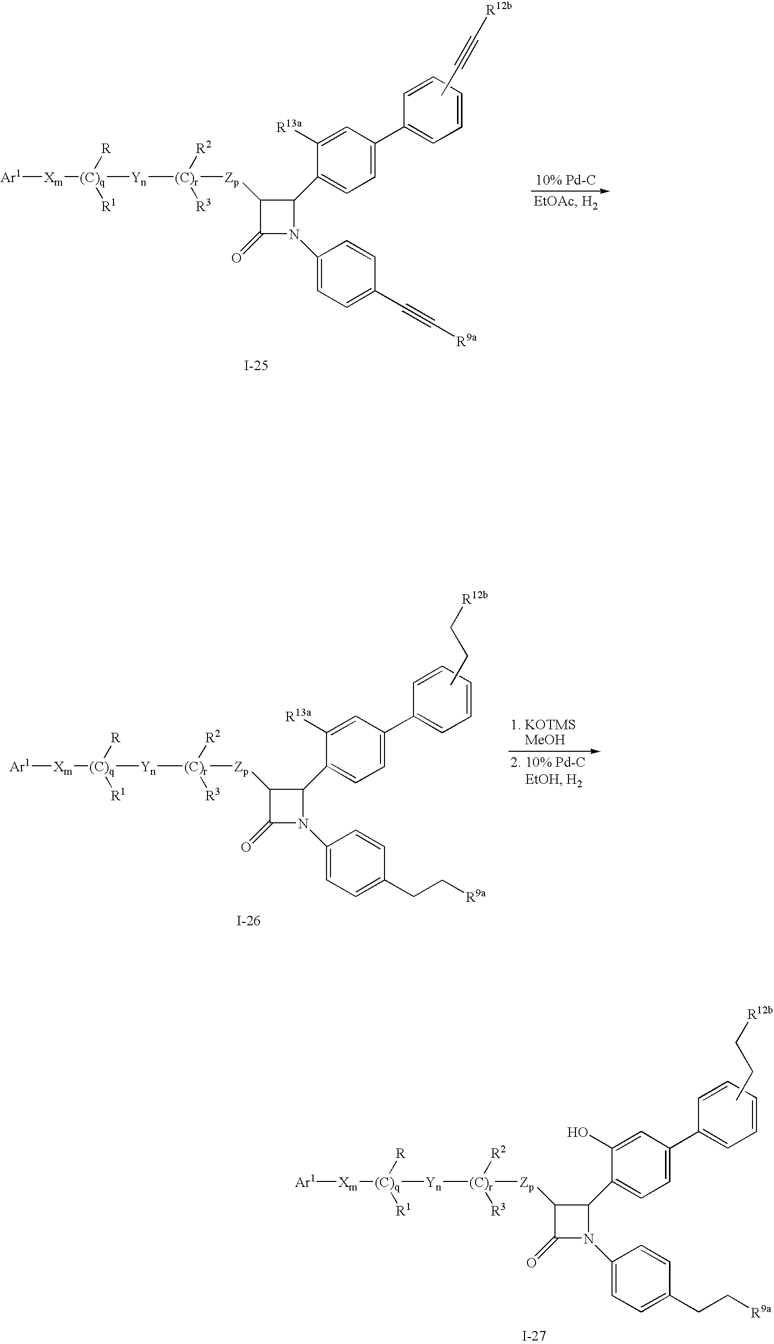

### Preparation of 4-bromo-2-hydroxybenzaldehyde (i-1)

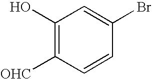

3-Bromophenol (10 g, 45 mmol) was dissolved in anhydrous acetonitrile (160 ml), cooled in an ice bath and magnesium chloride (12.8 g, 134 mmol) added portionwise over 10 mins. Triethylamine (25.3 ml, 363 mmol) was added to this mixture over 5 mins, followed by portionwise addition of paraformaldehyde (5.47 g, 636 mmol). After complete addition the mixture was heated at reflux for 18.5 hours. The mixture was cooled and quenched by the addition of sat. NH₄Cl (350 ml) and extracted with EtOAc (3×150 ml). The combined EtOAc layers were washed with sat. NaHCO₃ (2×150 mml), 1N HCl (2×150 ml), and sat. NaCl (2×100 ml), dried over Na₂SO₄, filtered and evaporated. The residue was purified by MPLC on silica gel eluting with a gradient rising from 100% hexanes to 20% EtOAc in hexanes. Product containing fractions were combined and evaporated and recrystallised from hot hexanes to give the title compound. ¹HNMR (500 MHz, CDCl₃) δ: 11.02 (s, 1H), 9.87 (s, 1H), 7.46 (d, 1H), 7.42 (dd, 1H), 7.25 (d, 1H).

### Preparation of 2-allyloxy-4-bromobenzaldehyde (i-2)

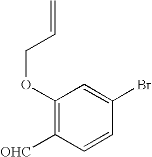

To a solution of 4-bromo-2-hydroxybenzaldehyde (i-1) (7.7 g, 38.0 mmol) in anhydrous DMF (50 mL) was added potassium carbonate (7.5 g, 54.3 mmol), followed by allyl bromide (5.0 mL, 57.8 mmol). The mixture was stirred at room temperature overnight. The reaction mixture was poured into water and extracted with ethyl acetate (3×100 mL) The extracts were combined, dried over magnesium sulfate, filtered and evaporated under vacuum. The residue was recrystallised from EtOAc/hexanes to give the title compound. ¹H-NMR (500 MHz, CDCl₃) δ: 10.55 (s, 1H), 7.56 (d, J=8 Hz, 1H), 7.43 (d, J=8 Hz, 1H), 7.35 (d, J=1 Hz, 1H), 6.10 (ddt, J=5.0, 11.5, 17.5 Hz, 1H), 5.48 (dq, J=1.5, 17.5 Hz, 1H), 5.40 (dq, J=1.5, 11.5 Hz, 1H), 4.67 (dt, 1.5, 5.0 Hz, 2H).

### Preparation of 4-({(1E)-[2-(allyloxy)-4-bromophenyl]methylene}amino)phenol (i-3)

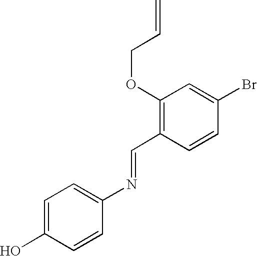

2-allyloxy-4-bromobenzaldehyde (i-2) (9.0 g, 37.3 mmol) was suspended in propan-2-ol (90 mL) and warmed until complete dissolution. 4-hydroxyaniline (4.1 g, 37.5 mmol) was added to the solution and the resulting mixture warmed at 50° C. for 4 hours. The cooled mixture was evaporated, and the residue triturated with a mixture of Et₂O and hexanes, filtered an air dried to give of the title compound. ¹H-NMR (500 MHz, D6-DMSO) δ: 8.77 (s, 1H), 7.56 (d, J=8.5 Hz, 2H), 7.51 (d, J=1.5 Hz, 1H), 7.44 (dd, J=1.5, 8.5 Hz, 1H) 7.30 (d, J=8.5 Hz, 1H), 7.17 (d, j=8.5 Hz, 2H), 6.06 (ddd, J=5.0, 10.5, 16.5 Hz, 1H), 5.40 (dq, J=1.5, 16.5 Hz, 1H), 5.28 (dq, 1.5, 10.5 Hz, 1H), 4.72 (dt, J=1.5, 5.0 Hz, 2H).

### Preparation of (4S)-3-{(2R,5S)-2-{(S)-[2-(allyloxy)-4-bromophenyl][(4-hydroyphenyl)amino]methyl}-5-(4-fluorophenyl)-5-[(trimethylsilyl)oxy]pentanoyl}-4-phenyl-1,3-oxazolidin-2-one (i-4)

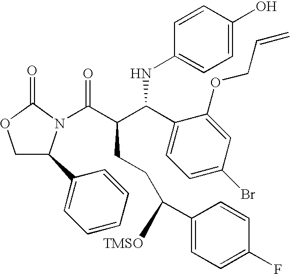

To a suspension of (4S)-3-[(5S)-5-(4-fluorophenyl)-5-hydroxypentanoyl]-4-phenyl-1,3-oxazolidin-2-one (251.6 g, 0.704 mol) (prepared according to the procedures of Fu, X.; McCallister, T. L.; Thiruvengadam, T. K.; Tann, C. H.; and Su, D. Tetrahedron Lett. (2003) 44, 801-804) and 4-({(1E)-[2-(allyloxy)-4-bromophenyl]methylene}amino)phenol (455 g, 1.41 mol; i-3) in CH₂Cl₂ (3.1 L) under nitrogen atmosphere at −5° C. was added N,N-diisoproplylethylamine (640 mL, 3.66 mol) keeping the temperature below 0° C. To the resulting suspension was added chlorotrimethylsilane (297 mL, 2.323 mol) keeping the temperature below 0° C. The resulting solution was stirred at −5° C. for 1 h at which time the reaction mixture was cooled to −30° C. To this cooled solution was added TiCl₄ (90 mL, 0.774 mol) keeping the temperature below −25° C. The resulting solution was stirred at −30° C. for 2.5 hrs at which time acetic acid (210 mL) was added keeping the temperature below −25° C. After the completion of the addition, the reaction mixture was poured into a pre-cooled 0° C. solution of potassium sodium tartrate (245 g) in water (3.5 L) cooled in an ice/salt bath. The resulting mixture was stirred at 0° C. for 1 hr at which time a solution of sodium hydrogensulfite (250 g) in water (1.25 L) was added. The resulting solution was stirred at ambient temperature overnight. Filter aid was added to the mixture; the reaction mixture was then filtered through a pad of filter aid. The solids were washed with CH₂Cl₂ and the filtrates transferred to a separatory funnel. The layers were separated and the aqueous layer extracted with CH₂Cl₂ (3 L). The combined organic layers were washed with water, dried over MgSO₄, filtered and the solvent removed under vacuum until ˜2 L of solution remained. This mixture was placed in a round bottom flask under nitrogen atmosphere and N,O-bis(trimethylsilyl)acetamide (216 mL, 0.866 mol) was added. After completion of the addition, the mixture was heated to 45° C., then kept at that temperature for 0.5 hr. The reaction mixture was cooled, concentrated under vacuum until a solid formed. A small amount of methyl-t-butylether was added followed by heptane (2 L). The resulting suspension was stirred for ten minutes, filtered and the resulting solid washed with heptane. The resulting solid was dried under vacuum at 60° C. overnight to afford the title compound, which was used without further purification. m/z (ES) 760 and 762 (M+H)⁺

### Preparation of (3R,4S)-4-[2-(allyloxy)-4-bromophenyl]-3-[(3S)-3-(4-fluorophenyl)-3-hydroxypropyl]-1-(4-hydroxyphenyl)azetidin-2-one (i-5)

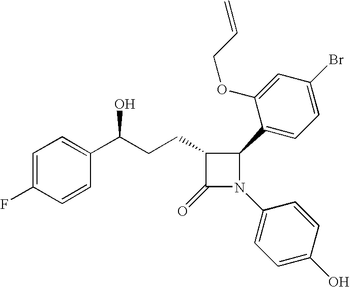

To a mixture of (4S)-3-{(2R,5S)-2-{(S)-[2-(allyloxy)-4-bromophenyl][(4-hydroyphenyl)amino]methyl}-5-(4-fluorophenyl)-5-[(trimethylsilyl)oxy]pentanoyl}-4-phenyl-1,3-oxazolidin-2-one (i-4) and 4-({(1E)-[2-(allyloxy)-4-bromophenyl]methylene}amino)phenol (i-3) in a suitable solvent such as methyl-t-butylether is added N,O-bis(trimethylsilyl)acetamide followed by tetra-n-butylammonium fluoride. The reaction mixture is stirred at ambient temperature for a time between 2 to 5 hours at which point acetic acid is added to the mixture and the resulting solution is stirred for a time between ten minutes to one hour. The solvent is evaporated under vacuum and the residue is dissolved in a suitable solvent such as 2-propanol. A solution of 1-5N H₂SO₄ is added to the mixture, and the resulting mixture is stirred at room temperature for a time between 5-24 h at which it is then poured into a separatory funnel containing water and a suitable organic solvent such as ethyl acetate or ether. The layers are separated and the aqueous layer is extracted further with the appropriately chosen organic solvent. The organic extracts are combined and then dried over MgSO₄, filtered and the solvent is removed under vacuum. The remaining residue is purified by MPLC or silica gel column chromatography with the appropriate gradient eluant of 0% to 80% ethyl acetate in a suitable non-polar solvent such as hexane or heptane which affords the title compound.

### Preparation of 4-{(2S,3R)-3-[(3S)-3-(acetyloxy)-3-(4-fluorophenyl)propyl]-2-[2-(allyloxy)-4-bromophenyl]-4-oxoazetidin-1-yl}phenyl acetate (i-6)

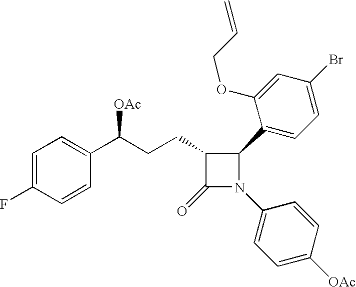

To a solution of the intermediate (3R,4S)-4-[2-(allyloxy)-4-bromophenyl]-3-[(3S)-3-(4-fluorophenyl)-3-hydroxypropyl]-1-(4-hydroxyphenyl)azetidin-2-one (i-5) in a suitable organic solvent such as dichloromethane is added an organic base such as anhydrous pyridine and acetic anhydride which are followed by the addition of a catalytic amount of DMAP. The reaction mixture is stirred at ambient temperature for a time between 1 to 5 hr at which time more pyridine and acetic anhydride are added to push the reaction to completion. The reaction mixture is stirred at ambient temperature for 16 to 24 hr and then is poured into a separatory funnel which contains a solution of 1 to 3N aqueous HCl. The layers are separated and the organic layer is washed with a saturated aqueous solution of sodium bicarbonate. The organic extracts are dried over magnesium sulfate, filtered, and the solvent is removed under vacuum. The residue is purified by MPLC or silica gel flash column chromatography eluting with an appropriate gradient of 0% to 80% of a polar solvent such as ethyl acetate in a non-polar solvent as hexane or heptane which affords the title compound.

### Preparation of 4-[(2S,3R)-3-[(3S)-3-(acetyloxy)-3-(4-fluorophenyl)propyl]-2-(4-bromo-2-hydroxyphenyl)-4-oxoazetidin-1-yl]phenyl acetate (i-7)

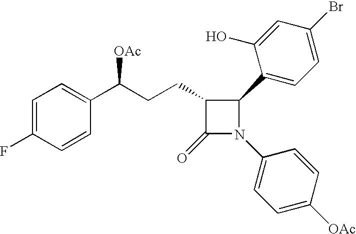

To a mixture of 4-{(2S,3R)-3-[(3S)-3-(acetyloxy)-3-(4-fluorophenyl)propyl]-2-[2-(allyloxy)-4-bromophenyl]-4-oxoazetidin-1-yl}phenyl acetate (i-6) in an appropriate organic solvent such as tetrahydrofuran under an inert atmosphere is added morpholine which is followed by the addition of tetrakistriphenylphosphine palladium. The resulting solution is flushed with an inert atmosphere like nitrogen or argon and the solution is stirred at room temperature for a time between 16 and 24 hours. The mixture is poured into an aqueous 1N solution of HCl and extracted with a suitable organic solvent such as ethyl acetate. The organic extracts are dried over a drying agent such as magnesium sulfate, filtered, and the filtrate is evaporated under vacuum. The residue is purified by MPLC or silica gel flask column chromatography with the appropriate organic eluant chosen.

### Preparation of 4-(2S,3R)-3-[(3S)-3-(acetyloxy)-3-(4-fluorophenyl)propyl]-2-[2-(benzyloxy)-4-bromophenyl]-4-oxoazetidin-1-yl}phenyl acetate (i-8)

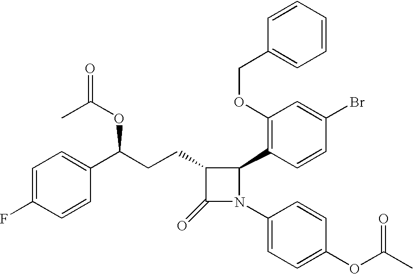

A solution of triphenylphosine and a suitable Mitsunobu reaction initiator such as DIAD or DEAD in a suitable solvent such as toluene or dioxane is flushed with an inert gas such as nitrogen or argon and cooled to 0° C. with an ice/water bath. To this solution is added dropwise via syringe a prepared solution of 4-[(2S,3R)-3-[(3S)-3-(acetyloxy)-3-(4-fluorophenyl)propyl]-2-(4-bromo-2-hydroxyphenyl)-4-oxoazetidin-1-yl]phenyl acetate (i-7) and benzoic acid in a similar solvent such as toluene or dioxane and the resulting mixture is stirred under inert atmosphere for a period of time between 16-48 hrs at which time the solution is allowed to warm to room temperature. The solution is evaporated under nitrogen and the resulting residue is purified by MPLC or silica gel flash column chromatography with the appropriate organic eluant which affords the title compound.

### Preparation of 4-(2S,3R)-3-[(3S)-3-(acetyloxy)-3-(4-fluorophenyl)propyl]-2-[2-(benzyloxy)-4-(4,4,5,5-tetramethyl-1,3,2-dioxaborolan-2-yl)phenyl]-4-oxoazetidin-1-yl]phenyl acetate (i-9)

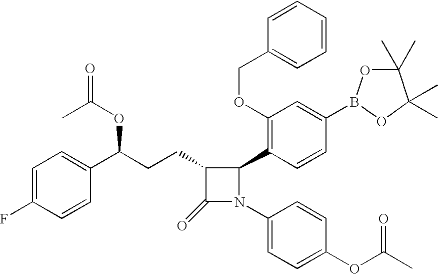

To a solution of 4-(2S,3R)-3-[(3S)-3-(acetyloxy)-3-(4-fluorophenyl)propyl]-2-[2-(benzyloxy)-4-bromophenyl]-4-oxoazetidin-1-yl}phenyl acetate (i-8) in a suitable solvent such as dioxane is added a suitable mild inorganic base such as potassium acetate and a boronating agent such as Bis(pinacolate)diboron, and the resulting solution is flushed with an inert gas and is stirred vigorously for a time between 1 and 5 minutes. A suitable palladium catalyst such as tetrakistriphenylphosphine palladium(0) or [1,1′-bis(diphenylphosphino)ferrocene]dichloropalladium(II), or the like, is added and the resulting mixture is flushed with an inert gas and heated between 40° C. and 100° C., for a period of 6-48 h. The mixture is poured into an organic solvent such as ethyl acetate and washed with an aqueous solution of 0.1 to 1 N HCl. The organic layer is dried over the appropriate drying agent, such as magnesium sulfate, filtered and the solvent is evaporated under vacuum. The material is used without any further purification.

### Preparation of (1S)-3-[(2S,3R)-2-[2-(benzyloxy)-4-bromophenyl]-4-oxo-1-(4-{[(trifluoromethyl)sulfonyl]oxy}phenyl)azetidin-3-yl]-1-(4-fluorophenyl)propyl acetate (i-10)

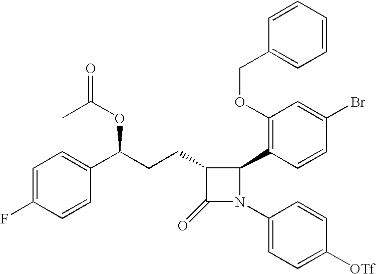

To a solution of 4-(2S,3R)-3-[(3S)-3-(acetyloxy)-3-(4-fluorophenyl)propyl]-2-[2-(benzyloxy)-4-bromophenyl]-4-oxoazetidin-1-yl}phenyl acetate (i-8) in a suitable solvent such as MeOH is added guanidine which is followed by a suitable organic base such as TEA. The resulting mixture is stirred at room temperature for a period of time between 1-8 hrs at which time the solvent is removed under vacuum. The residue is tritrated between an appropriate solvent such as ethyl acetate or ether and 1N aq. HCl. The layers are separated and the organic layer is washed with brine, dried over a suitable drying agent such as MgSO₄, filtered and the solvent is removed under vacuum.

The material obtained above is dissolved in dichloromethane and then is treated with trifluoromethanesulfonic anhydride in the presence of an appropriate base such as pyridine. The reaction mixture is stirred for a period of time between 1-8 hrs. The reaction mixture is washed with aqueous hydrochloric acid and then brine, dried over a suitable drying agent such as anhydrous MgSO₄ powder, filtered, and the solvent is evaporated under reduced pressure. The residue is purified by MPLC or silica gel flash column chromatography eluting with the appropriate organic solvents which affords the title compound.

Intermediates related to those described above in i-9 and i-10 of varying 2-substitution on the phenyl ring, such as H or F, are prepared from the appropriate starting materials using the procedures described above.

### Preparation of α-D-gluocopyranosyl bromide, tetraacetate (i-11)

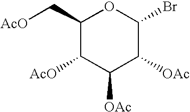

The compound α-D-gluocopyranosyl bromide, tetraacetate (i-11) was prepared according to the procedure in Kartha, K. P. R.; Jennings, H. J.; J. Carbohydr. Chem. (1990), 9, 777-781 from the commercially available starting material α-D-Glucopyranose.

### Preparation of (4-bromophenyl)magnesium (i-12)

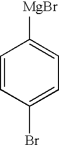

A 3-necked, 5000 mL round bottom flask equipped with a magnetic stirrer, thermocouple, nitrogen bubbler, and addition funnel was charged with magnesium turnings (12 g, 0.49 mol) and 500 mL ether. 1,4-dibromobenzene (114 g, 0.48 mol) was dissolved in 500 mL ether and placed in the addition funnel. About 175 mL of the solution was added, but no increase in temperature was seen. About 0.4 mL of 1,2 dibromoethane was added to initiate the reaction. The reaction was then heated to reflux and the temperature seemed to remain constant at 35° C. The rest of the 1,4-dibromobenzene solution was added slowly dropwise and the mixture was allowed to reflux for 30 minutes after the full addition was complete. The mixture was allowed to cool to room temperature once the reaction was complete. The mixture is used without purification or isolation for further reactions.

### Preparation of (3-bromophenyl)magnesium (i-13) and (2-bromophenyl)magnesium (i-14)

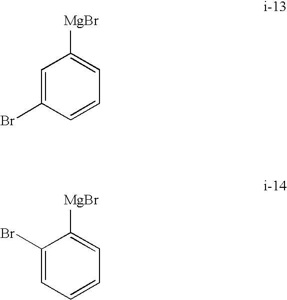

The title compounds, (3-bromophenyl)magnesium (i-13) and (2-bromophenyl)magnesium (i-14) were prepared employing the procedure described in the preparation of i-12 with the appropriate dibromobenzene starting material.

### Preparation of (2R,3R,4R,5S,6S)-2-[(acetyloxy)methyl]-6-(4-bromophenyl)tetrahydro-2H-pyran-3,4,5-trityl triacetate (i-15) and (2R,3R,4R,5S,6S)-2-[(acetyloxy)methyl]-6-(3-bromophenyl)tetrahydro-2H-pyran-3,4,5-trityl triacetate (i-16)

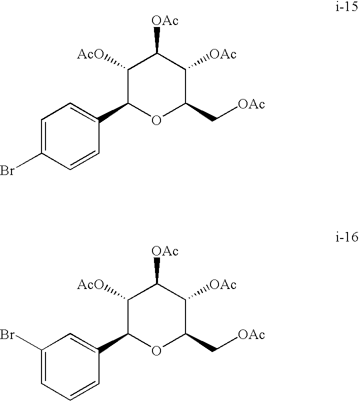

The compounds (2R,3R,4R,5S,6S)-2-[(acetyloxy)methyl]-6-(4-bromophenyl)tetrahydro-2H-pyran-3,4,5-trityl triacetate (i-15) and (2R,3R,4R,5S,6S)-2-[(acetyloxy)methyl]-6-(3-bromophenyl)tetrahydro-2H-pyran-3,4,5-trityl triacetate (i-16) were prepared according to the procedure in Panigot, M. J.; Humphries, K. A.; Curley, R. W. J. J. Carbohydr Chem. (1994), 13, 303-321 from the intermediates i-11, i-12, and i-13, described previously. m/z (ES) 486 and 488 (M+H)⁺ (found for both i-15 and i-16).

### Preparation of 5-(4-bromophenyl)-2,2-dimethyl-1,3-dioxan-5-ol (i-17)

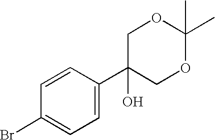

A round bottom flask is charged with magnesium turnings and a suitable organic solvent such as ether and the mixture set under inert atmosphere. 1,4-dibromobenzene is dissolved in the same organic medium as the mixture set under inert atmosphere and this solution is added via an addition funnel dropwise over a period of time between 1-30 minutes. A catalytic amount of 1,2 dibromoethane is added to initiate the reaction. The reaction is heated to reflux and left for a period of time between 30 minutes to 1 hour. The mixture is then allowed to cool to room temperature slowly at which time the magnesium gridnard should be ready. A solution of 2,2-dimethyl-1,3-dioxan-5-one in the appropriate solvent is added to the mixture via addition funnel dropwise and the resulting solution is stirred for a period of time between 5-15 hrs at room temperature to 35° C. The mixture is then allowed to age overnight at ambient temperature. The solution is poured slowly over a solution of 10:1 water and acetic acid and extracted with the appropriate organic solvent such as ether. The extracts are dried over a suitable drying agent such as sodium sulfate, filtered, and the solvent is removed under vacuum. The residue is purified by MPLC or silica gel flash column chromatography with the appropriate organic eluant which affords the title compound.

### Preparation of 5-(3-bromophenyl)-2,2-dimethyl-1,3-dioxan-5-ol (i-18)

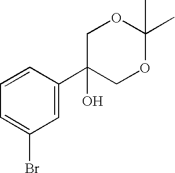

The title compound, 5-(3-bromophenyl)-2,2-dimethyl-1,3-dioxan-5-ol, is prepared as described in the preparation of (i-17) from the appropriate dibromobenzene starting material.

### Preparation of 5-(2-bromophenyl)-2,2-dimethyl-1,3-dioxan-5-ol (i-19)

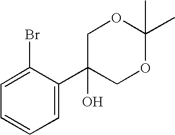

The title compound, 5-(2-bromophenyl)-2,2-dimethyl-1,3-dioxan-5-ol, is prepared as described in the preparation of (i-17) from the appropriate dibromobenzene starting material.

### Preparation of 5-ethynyl-2,2-dimethyl-1,3-dioxan-5-yl acetate (i-20)

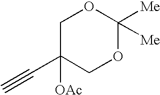

To a dry 250 mL roundbottom flask was charged a 0.5M solution of ethynylmagnesium bromide in THF (115 mL, 57.7 mmol) under nitrogen atmosphere. The resulting solution was cooled to 0° C. in an ice bath. To the cooled solution was added slowly a solution of 2,2-dimethyl-1,3-dioxane-5-one (5 g, 38.44 mmol) in 50 m/L dry THF. The ice bath was removed and the resulting reaction mixture was stirred at ambient temperature for 1.5 hrs. The reaction mixture was quenched with sat. aq. NH₄Cl (50 mL) and then extracted with ethyl acetate (100 mL). The organic layer was dried over Na₂SO₄, filtered and the solvent removed under vacuum to afford the crude intermediate.

The crude intermediate was dissolved in CH₂Cl₂ (100 mL) under nitrogen atmosphere. To the resulting solution was added simultaneously by syringe acetic anhydride (4.34 mL, 46 mmol) and TEA (6.4 mL, 46 mmol). To the reaction mixture was added DMAP (0.56 g, 4.6 mmol). The reaction mixture was stirred for 3 hrs at room temperature at which time the reaction was quenched by the addition of 1N aq. HCl (100 mL). The reaction mixture was transferred to separatory funnel and the organic layer was separated. The organic layer was washed with aq. NaHCO₃ (100 mL), water (50 mL), brine, dried, filtered and the solvent removed under vacuum to afford the title compound (i-20) which was used without further purification. ¹HNMR (500 MHz, CDCl₃) δ: 4.14 (d, J=12.6, 2H) 4.07 (d, J=12.6 Hz, 2H), 2.65 (s, 1H), 2.12 (s, 3H), 1.45 (s, 3H), 1.41 (s, 3H).

### Preparation of 2-ethynylpropane-1,2,3-triol 1,3-diacetate (i-21)

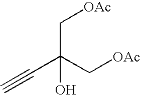

The related intermediate i-21 was prepared from 1,3-diacetoxyacetone using a procedure similar to that described in the first paragraph for i-20 above. ¹H-NMR (400 MHz, CDCl₃) δ: 4.28 (d, J=11.5 Hz, 2H), 4.22 (J=11.5 Hz, 2H), 3.26 (s, 1H), 2.55 (s, 1H), 2.13 (s, 6H).

### Preparation of N-prop-2-yn-1-ylmethanesulfonamide (i-22)

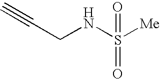

Methansulfonylchloride (1.40 mL, 18.1 mmol) was added dropwise to a stirred solution of propargylamine (1.00 g, 18.1 mmol) and dimethylaminopyridine (44.0 mg, 0.36 mmol) in pyridine (10 mL) at 0° C. After aging for approximately 15 h, the reaction mixture was poured into 1N HCl and extracted twice with ethyl acetate. The combined organic extracts were washed with saturated aqueous sodium bicarbonate, brine, dried (MgSO₄), filtered and concentrated in vacuo, to afford the title compound i-22. Crude i-22 crystallized on standing and was used without further purification. ¹HNMR (500 MHz, CDCl₃) δ: 4.92 (br s, 1H), 3.99 (dd, J=2.3, 6.2 Hz, 2H), 3.11 (s, 3H), 2.70 (br t, J=2.3 Hz).

### Preparation of N-Methyl-N-prop-2-yn-1-ylmethanesulfonamide (i-23)

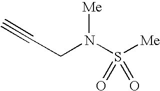

Methanesulfonylchloride (1.12 mL, 14.5 mmol) was added to a stirred solution of N-methylpropargylamine (1.22 mL, 14.5 mmol) and dimethylaminopyridine (35 mg, 0.30 mmol) in pyridine (10 mL) at room temperature. After aging for approximately 15 h, the reaction mixture was poured into ethyl acetate and washed successively with 1N HCl and brine. The organic phase was dried (Na₂SO₄), filtered and concentrated in vacuo, to afford the title compound (i-23), which was used without further purification. (500 MHz, CDCl₃) δ: 4.96 (br s, 1H), 4.01 (dd, J=2.4, 6.7 Hz, 2H), 3.11 (s, 3H), 2.66 (br t, J=2.4 Hz), 2.42 (s, 3H).

### Preparation of [(hex-5-yn-1-yloxy)methyl]benzene or benzyl hex-5-yn-1-yl ether (i-24)

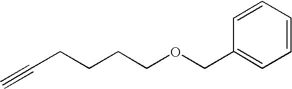

To a solution of 5-hexyn-1-ol (1.17 g, 11.88 mmol) in anhydrous DMF (100 mL) under nitrogen atmosphere was added TBAI (0.87 g, 2.38 mmol) followed by 60% NaH dispersion in oil (0.55 g, 14.26 mmol) in portions over 0.5 h. The reaction mixture was stirred for 0.5 hr at which time benzyl bromide (2.44 g, 14.26 mmol) was added by syringe. The reaction mixture was stirred for 16 h at room temperature at which time the reaction was quenched by the addition of sat. aq. NH₄Cl (100 mL). The reaction mixture was transferred to separatory funnel and extracted with ether (3×75 mL). The combined organic extracts were washed with water (50 mL), brine (75 mL), dried (Na₂SO₄), filtered and the solvent removed under vacuum. The residue was purified by MPLC (silica column) with stepwise gradient elution (0-60% EtOAc/hexanes as eluent) to afford the title compound (i-24).

m/z (ES) 211 (M+Na)⁺

Intermediates related to those described above of varying substitution and chain length may be prepared from the appropriate starting materials using the procedures described above.

### Preparation of 4-(methylsulfonyl)but-1-yne (i-25)

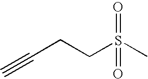

A solution of 3-butyn-1-ol (1000 mg; 14.27 mmol) and methanesulfonyl chloride (1.63 g, 14.27 mmol) in dichloromethane (35 ml) was cooled in a bath to 0° C. and to this solution triethylamine (2.09 ml, 14.98 mmol) in dichloromethane (5 ml) was added drop by drop over about 5 minutes. The resulting reaction mixture was stirred vigorously for 0.5 h at 0° C. and then the stirring was continued for a further 0.5 h at room temperature. The volatiles were removed on a rotary evaporator under reduced pressure and the residues left were partitioned between diethyl ether (2×50 ml) and 1N hydrochloric acid (50 ml). The combined ethereal extracts were dried over anhydrous magnesium sulfate powder, filtered and the resulting filtrates concentrated under reduced pressure to leave a liquid which was the but-3-yn-1-yl methanesulfonate ester.

To a solution of the crude but-3-yn-1-yl methanesulfonate ester (0.5 g, 3.37 mmol) in ethanol (7.5 ml) was added sodium thiomethoxide (248 mg, 3.54 mmol) powder in small batches over about 5 minutes and the resulting mixture stirred under an inert atmosphere for 12 h at room temperature. A few drops of distilled water were added to dissolve up the cloudy solution and give a solution. A peracetic acid solution was prepared from 30% aqueous hydrogen peroxide (3 ml), acetic acid (5 ml) and 3 drops of conc. sulfuric acid at 0° C. A portion of this peracid solution (3 ml) was added cautiously to the ethanol solution and the reaction mixture was stirred at room temperature for 8 h, then concentrated on a rotary evaporator and the oily residues obtained were partitioned with dichloromethane (3×25 ml) and water. The combined dichloromethane extracts were washed with saturated sodium carbonate solution added to neutralize the acid (tested with pH paper) and with saturated sodium sulfite solution to remove excess oxidant (until negative to starch iodide paper). The dichloromethane layer was dried over anhydrous magnesium sulfate powder, filtered and the filtrates concentrated under reduced pressure. The oil which remained on evaporation was purified on preparative tlc plates that were eluted with dichloromethane:Methanol (97:3 v/v) to give the product 4-(methylsulfonyl)but-1-yne. ¹H-NMR (400 MHz, CD₃OD) δ: 3.19 (t, J=7 Hz, 2H), 2.98 (s, 3H), 2.74 (dt, J=7, 2.5 Hz, 2H), 2.13 (t, J=2.5 Hz, 1H).

These intermediates related to those described above of varying substitution and chain length may be prepared from the appropriate starting materials using the procedures described above for i-25.

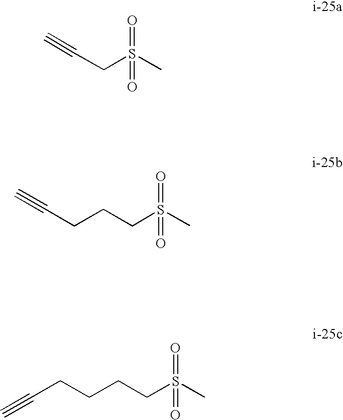

### Preparation of dibenzyl ethynylmalonate (i-26)

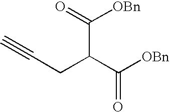

To a solution of dibenzyl malonate (2 g, 7.03 mmol) in anhydrous DMF/THF (1:1 solution, 50 mL) set under nitrogen atmosphere and cooled to 0° C. was added in portions sodium hydride (NaH, 60% in oil, 310 mg) and the resulting mixture stirred for one hour at 0° C. To this mixture was then added propargyl bromide (0.63 mL, 7.03 mmol) via syringe and the resulting mixture stirred for 3 hours allowing to warm to room temperature. The reaction was quenched with aqueous saturated solution of ammonium chloride and extract with ether (3×50 mL). The organics were combined and washed with water (50 mL), followed by brine (50 mL). The organics were dried over sodium sulfate, filtered and concentrated in vacuo. Horizon MPLC purification using a gradient eluant of 0-60% ethyl acetate in hexane afforded the title compound. ¹HNMR (500 MHz, CDCl₃) δ: 7.40-7.31 (m, 10H), 5.21 (s, 4H), 3.73 (t, J=7.5 Hz, 1H), 3.51 (s, 1H), 2.86 (dd, J=2.6, 7.6 Hz, 2H).

### Preparation of 1-prop-2-yn-1-yl-1H-1,2,4-triazole (i-27)

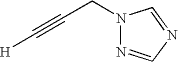

To a solution of 1H-1,2,4-triazole (5 g, 72.4 mmol) in ethanol (50 mL) cooled in a ice-bath was added solution of NaOH (2.9 g, 74.7 mmol) in 5 mL water which immediately resulted in the formation of a white precipitate. To the resulting mixture was added dropwise over 1 h propargyl bromide (8.2 mL, 74.7 mmol). After completion of the addition, the reaction mixture was allowed to warm to RT and stirred for 48 hr. Water (100 mL) was added and the reaction mixture was transferred to a separatory funnel and extracted with methylene chloride (3×75 mL). The combined organic layers were washed with water (2×), dried over Na₂SO₄ filtered and the solvent removed under vacuum. The residue was purified by column chromatography on silica gel eluting with 2% MeOH in CH₂Cl₂ to provide of the title compound. ¹H NMR (500 MHz, CDCl₃) δ: 8.29 (s, 1H), 7.96 (s, 1H), 4.99 (d, J=2.7, 2H), 2.60 (t, J=2.7, 1H)

### Preparation of 3-Iodo-1-trityl-1H-1,2,4-triazole (i-28)

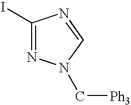

3-Iodo-1-trityl-1H-1,2,4-triazole (i-28) was prepared according to the procedure described in PCT publication (WO 93/15610 A1). ¹HNMR (500 MHz, CDCl₃) δ: 8.09 (s, 1H), 7.38 (m, 9H), 7.04 (m, 6H).

### Preparation of 3-(1-trimethylsilylethyn-2-yl)-1-trityl-1H-1,2,4-triazole (i-29)

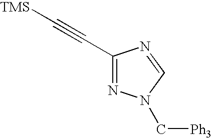

Nitrogen gas was bubbled through a solution of 3-iodo-1-trityl-1H-1,2,4-triazole (i-28, 37.3 g, 85.35 mmol) and triethylamine (17.8 ml, 128 mmol) in anhydrous DMF (300 ml) heated at 35° C. for 30 mins. Pd(PPh₃)₂Cl₂ (2.4 g, 3.4 mmol) and CuI (651 mg, 3.4 mmol) were added followed by addition of ethynyltrimethylsilane (18 ml, 128 mmol) in anhydrous DMF (18 ml) over 15 hours via syringe pump. After complete addition the mixture was heated at 35° C. for a further 5 hours. The mixture was poured into water (700 ml) and extracted with EtOAc (3×300 ml). Combined EtOAc layers were washed with water (2×500 ml), sat. NaCl (250 ml), dried over Na₂SO₄, filtered and evaporated. The residue was purified by MPLC on silica gel eluting with a gradient from 100% hexanes to 10% EtOAc in hexanes to afford the title compound. ¹HNMR (500 MHz, CDCl₃) δ: 7.96 (s, 1H), 7.37 (m, 9H), 7.14 (m, 6H), 0.27 (s, 9H).

### Preparation of 3-ethynyl-1-trityl-1H-1,2,4-triazole (i-30)

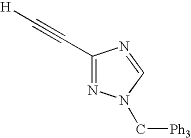

Tetrabutylammonium fluoride (3.8 ml of a 1.0M solution in THF, 3.8 mmol) was added to a solution of 3-(1-trimethylsilylethyn-2-yl)-1-trityl-1H-1,2,4-triazole (i-29, 7.75 g, 19 mmol) in anhydrous THF (50 ml), and the resulting mixture stirred for 30 mins, evaporated to dryness, and the residue partitioned between CH₂Cl₂ and water. The organic layer was washed with sat. NaCl, dried over Na₂SO₄, filtered and evaporated. The residue was triturated with Et₂O/hexanes to afford of the title compound. ¹HNMR (500 MHz, CDCl₃) δ: 7.99 (s, 1H), 7.38 (m, 9H), 7.15 (m, 6H), 3.10 (s, 1H).

### Preparation of 1-prop-2-en-1-yl-1H-1,2,3-triazole (i-31)

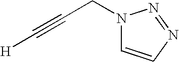

The title compound was prepared from 1H-1,2,3-triazole according to the procedure for intermediate (i-27). ¹H NMR (500 MHz, CDCl₃) δ: 7.80 (s, 1H), 7.74 (s, 1H), 5.22 (d, J=2.5, 2H), 2.59 (t, J=2.5, 1H)

### Preparation of (1S)-3-[(2S,3R)-2-[4-allyl-2-(benzyloxy)phenyl]-4-oxo-1-(4-{[(trifluoromethyl)sulfonyl]oxy}phenyl)azetidin-3-yl]-1-(4-fluorophenyl)propyl acetate (i-32)

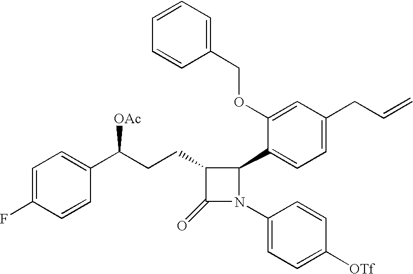

To a solution of (1S)-1-(4-fluorophenyl)-3-[(2S,3R)-2-[4-bromo-2-(benzyloxy)phenyl]-4-oxo-1-(4-{[(trifluoromethyl)sulfonyl]oxy}phenyl)azetidin-3-yl]propyl acetate (i-10) in a suitable solvent such as anhydrous dioxane is added lithium chloride and palladium tetrakis and the resulting solution is set under inert atmosphere, such as nitrogen or argon. Allyl tributyltin is then added to the solution via syringe and the resulting mixture is heated to a temperature between 40-65° C. for a period of time between 5-16 hours. The reaction is cooled to room temperature and the solution is evaporated in vacuo. The residue is dissolved in an appropriate organic solvent such as ethyl acetate and is washed with water and brine. The organic extracts are dried over a suitable drying agent such as magnesium sulfate, filtered, and are evaporated in vacuo. MPLC purification or silica gel flash column chromatography using an appropriate organic eluant affords the title compound.

Intermediates related to those described above (i-32) of varying substitution and chain length are prepared from the appropriate starting materials using the procedure described above.

### Preparation of 2,2-dimethyl-5-(4-vinylphenyl)-1,3-dioxan-5-ol (i-33)

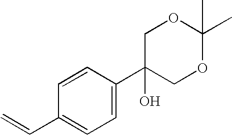

To a solution of 5-(4-bromophenyl)-2,2-dimethyl-1,3-dioxan-5-ol (i-17) in a suitable solvent such as anhydrous dioxane is added lithium chloride and tetrakistriphenylphosphine palladium and the resulting solution is set under inert atmosphere, such as nitrogen or argon. Vinyl tributyltin is then added to the solution via syringe and the resulting mixture is heated to a temperature between 40-65° C. for a period of time between 5-16 hours. The reaction is cooled to room temperature and the solution is evaporated in vacuo. The residue is dissolved in an appropriate organic solvent such as ethyl acetate and is washed with water and brine. The organic extracts are dried over a suitable drying agent such as magnesium sulfate, filtered, and are evaporated in vacuo. MPLC purification or silica gel flash column chromatography using an appropriate organic eluant affords the title compound.

Intermediates related to those described above (i-33) of varying substitution; such as the sugar or protected hydroxyl; various substitution positions; such as ortho, meta, or para; and chain length are prepared from the appropriate starting materials using the procedure described above.

### EXAMPLE 1

### N-[3-(4-{(2S,3R)-2-{4′-[1,2-dihydroxy-1-(hydroxymethyl)ethyl]-3-hydroxybiphenyl-4-yl}-3-[(3S)-3-(4-fluorophenyl)-3-hydroxypropyl]-4-oxoazetidin-1-yl}phenyl)propyl]methanesulfonamide

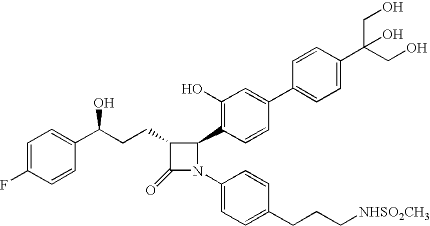

### Step A: 4-{(2S,3R)-3-[(3S)-3-(acetyloxy)-3-(4-fluorophenyl)propyl]-2-[3-(benzyloxy)-4′-(5-hydroxy-2,2-dimethyl-1,3-dioxan-5-yl)biphenyl-4-yl]-4-oxoazetidin-1-yl}phenyl acetate

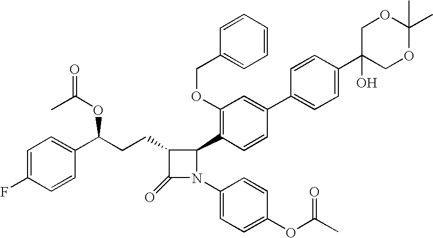

To a solution of 5-(4-bromophenyl)-2,2-dimethyl-1,3-dioxan-5-ol (i-17) in a suitable solvent system like toluene and ethanol is added 4-(2S,3R)-3-[(3S)-3-(acetyloxy)-3-(4-fluorophenyl)propyl]-2-[2-(benzyloxy)-4-(4,4,5,5-tetramethyl-1,3,2-dioxaborolan-2-yl)phenyl]-4-oxoazetidin-1-yl}phenyl acetate (i-9) and the solution is set under inert atmosphere such as nitrogen or argon. A mild base such as triethylamine or a solution of potassium carbonate is added to the mixture via a syringe or addition funnel followed by a suitable palladium catalyst such as tetrakistriphenylphosphine palladium, and the resulting mixture is heated to reflux for a period of time between 16 and 48 hr. The mixture is poured over water and extracted with the appropriate solvent such as ethyl acetate. The organic extracts are dried over a suitable drying agent as magnesium sulfate, filtered and the solvent is removed under vacuum. Purification by MPLC or silica gel flash column chromatography eluting with an appropriate solvent system affords the title compound.

### Step B: (1S)-3-[(2S,3R)-2-[3-(benzyloxy)-4′-(5-hydroxy-2,2-dimethyl-1,3-dioxan-5-yl)biphenyl-4-yl]-4-oxo-1-(4-{[(trifluoromethyl)sulfonyl]oxy}phenyl)azetidin-3-yl]-1-(4-fluorophenyl)propyl acetate

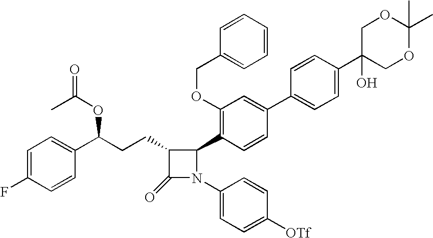

To a solution of 4-{(2S,3R)-3-[(3S)-3-(acetyloxy)-3-(4-fluorophenyl)propyl]-2-[3-(benzyloxy)-4′-(5-hydroxy-2,2-dimethyl-1,3-dioxan-5-yl)biphenyl-4-yl]-4-oxoazetidin-1-yl}phenyl acetate (Example 1, Step A) in a suitable solvent such as MeOH is added guanidine which is followed by a suitable organic base such as TEA. The resulting mixture is stirred at room temperature for a period of time between 1-8 hrs at which time the solvent is removed under vacuum. The residue is tritrated between an appropriate solvent such as ethyl acetate or ether and 1N aq. HCl. The layers are separated and the organic layer is washed with brine, dried over a suitable drying agent such as MgSO₄, filtered and the solvent is removed under vacuum.

The material obtained above is dissolved in dichloromethane and then is treated with trifluoromethanesulfonic anhydride in the presence of an appropriate base such as pyridine. The reaction mixture is stirred for a period of time between 1-8 hrs. The reaction mixture is washed with aqueous hydrochloric acid and then brine, dried over a suitable drying agent such as anhydrous MgSO₄ powder, filtered, and the solvent is evaporated under reduced pressure. The residue is purified by MPLC or silica gel flash column chromatography eluting with the appropriate organic solvents, affording the title compound.

### Step C: (1S)-3-[(2S,3R)-2-[3-(benzyloxy)-4′-(5-hydroxy-2,2-dimethyl-1,3-dioxan-5-yl)biphenyl-4-yl]-1-(4-{3-[(methylsulfonyl)amino]prop-1-yn-1-yl}phenyl)-4-oxoazetidin-3-yl]-1-(4-fluorophenyl)propyl acetate

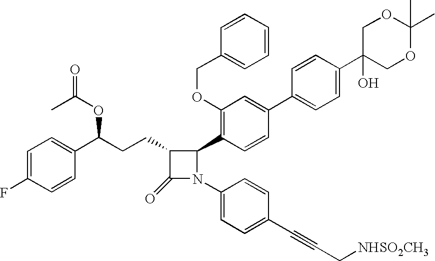

A solution of (1S)-3-[(2S,3R)-2-[3-(benzyloxy)-4′-(5-hydroxy-2,2-dimethyl-1,3-dioxan-5-yl)biphenyl-4-yl]-4-oxo-1-(4-{[(trifluoromethyl)sulfonyl]oxy}phenyl)azetidin-3-yl]-1-(4-fluorophenyl)propyl acetate (Example 1, step B) in an appropriate solvent such as DMF is then treated with a terminal alkyne of type i-22 in the presence of a suitable palladium catalyst such as tetrakistriphenylphosphine palladium(0) or [1,1′-bis(diphenylphosphino)ferrocene]dichloropalladium(II) or the like, and copper(I) iodide and an initiator such as tetra-n-butylammonium iodide. The reaction is heated between 40° C. and 100° C., for a period of 6-48 h, under an inert atmosphere such as nitrogen and argon. The cooled mixture is tritrated between a suitable organic solvent such as ethyl acetate and a solution of 0.5-2N hydrochloric acid. The organics are dried over an appropriate drying agent such as sodium sulfate, filtered, and the solvent is removed under vacuum. The residue is purified by MPLC or silica gel flash column chromatography eluting with the appropriate solvent system, affording the title compound.

### Step D: (1S)-3-[(2S,3R)-2-[3-(benzyloxy)-4′-(5-hydroxy-2,2-dimethyl-1,3-dioxan-5-yl)biphenyl-4-yl]-1-(4-{3-[(methylsulfonyl)amino]propyl}phenyl)-4-oxoazetidin-3-yl]-1-(4-fluorophenyl)propyl acetate

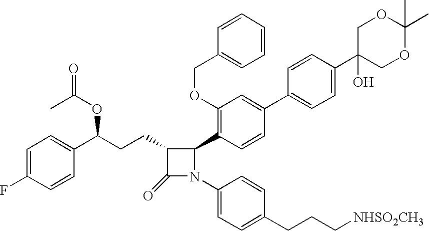

A round bottom flask is charged with 10% palladium on carbon, and a solution of (1S)-3-[(2S,3R)-2-[3-(benzyloxy)-4′-(5-hydroxy-2,2-dimethyl-1,3-dioxan-5-yl)biphenyl-4-yl]-1-(4-{3-[(methylsulfonyl)amino]prop-1-yn-1-yl}phenyl)-4-oxoazetidin-3-yl]-1-(4-fluorophenyl)propyl acetate (Example 1, step C) in a suitable solvent such as ethyl acetate is added to the catalyst. The mixture is set under hydrogen atmosphere and is stirred vigorously for a period of time between 1-24 hrs. The catalyst is removed by filtration through celite and the solvent is evaporated under vacuum. Purification by MPLC or silica gel flash column chromatography eluting with an appropriate solvent system affords the title compound.

### Step E: (1S)-3-[(2S,3R)-2-{3-(benzyloxy)-4′-[1,1,2-dihydroxy-1-(hydroxymethyl)ethyl]biphenyl-4-yl}-1-(4-{3-[(methylsulfonyl)amino]propyl}phenyl)-4-oxoazetidin-3-yl]-1-(4-fluorophenyl)propyl acetate

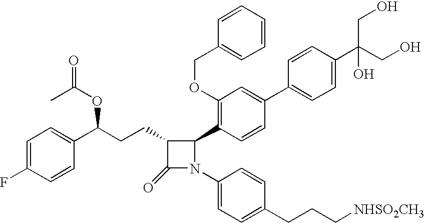

To a solution of (1S)-3-[(2S,3R)-2-[3-(benzyloxy)-4′-(5-hydroxy-2,2-dimethyl-1,3-dioxan-5-yl)biphenyl-4-yl]-1-(4-{3-[(methylsulfonyl)amino]propyl}phenyl)-4-oxoazetidin-3-yl]-1-(4-fluorophenyl)propyl acetate (Example 1, Step D) in an appropriate solvent such as tetrahydrofuran is added an anhydrous acid such as trifluoroacetic acid, and the resulting mixture is stirred at ambient temperature for a period of time between 2-16 hours. The solvent is removed under vacuum and the residue is used without further purification.

### Step F: Preparation of N-[3-(4-{(2S,3R)-2-{3-(benzyloxy)-4′-[1,2-dihydroxy-1-(hydroxymethyl)ethyl]biphenyl-4-yl}-3-[(3S)-3-(4-fluorophenyl)-3-hydroxypropyl]-4-oxoazetidin-1-yl}phenyl)propyl]methanesulfonamide

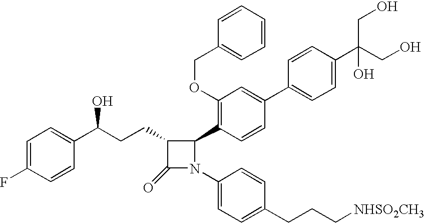

To a solution of (1S)-3-[(2S,3R)-2-{3-(benzyloxy)-4′-[1,2-dihydroxy-1-(hydroxymethyl)ethyl]biphenyl-4-yl}-1-(4-{3-[(methylsulfonyl)amino]propyl}phenyl)-4-oxoazetidin-3-yl]-1-(4-fluorophenyl)propyl acetate (Example 1; Step E) in a suitable solvent such as EtOH is added a mild base such as potassium trimethylsilanoate or potassium cyanide, and the resulting mixture is stirred at room temperature for 2-16 hrs or heated to 50° C. for 1-3 hours. To the reaction mixture is then added one equivalent of an anhydrous acid such as 2N HCl in ether to quench the base. The mixture is then filtered and purified by prep HPLC using a reverse phase column such as a C-18 Sunfire column eluting with gradient CH₃CN/0.1% aq. TFA (5 to 90%) in water. The product fractions are collected and freeze dried from CH₃CN/water, affording the title compound.

### Step G: Preparation of N-[3-(4-{(2S,3R)-2-{4′-[1,2-dihydroxy-1-(hydroxymethyl)ethyl]-3-hydroxybiphenyl-4-yl}-3-[(3S)-3-(4-fluorophenyl)-3-hydroxypropyl]-4-oxoazetidin-1-yl}phenyl)propyl]methanesulfonamide

A round bottom flask is charged with 10% palladium on carbon, and a solution of N-[3-(4-{(2S,3R)-2-{3-(benzyloxy)-4′-[1,2-dihydroxy-1-(hydroxymethyl)ethyl]biphenyl-4-yl}-3-[(3S)-3-(4-fluorophenyl)-3-hydroxypropyl]-4-oxoazetidin-1-yl}phenyl)propyl]methanesulfonamide (Example 1, step F) in a suitable alcohol solvent such as ethanol is added to the catalyst. The mixture is set under hydrogen atmosphere and stirred vigorously for a period of time between 1-24 hrs. The catalyst is removed by filtration through celite and the solvent is evaporated under vacuum. Purification is performed by prep HPLC using a reverse phase column such as a C-18 Sunfire column eluting with gradient CH₃CN/0.1% aq. TFA (5 to 90%) in water. The product fractions are collected and freeze dried from CH₃CN/water, affording the title compound.

### EXAMPLE 2

### (3R,4S)-3-[(3S)-3-(4-fluorophenyl)-3-hydroxypropyl]-4-{3-hydroxy-3′-[(2S,3R,4R,5S,6R)-3,4,5-trihydroxy-6-(hydroxymethyl)tetrahydro-2H-pyran-2-yl]biphenyl-4-yl}-1-{4-[2-(1H-1,2,4-triazol-3-yl)ethyl]phenyl)azetidin-2-one

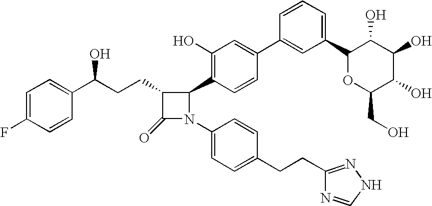

### Step A: Preparation of (2S,3S 4R,5R,6R)-2-[4′-{(2S,3R)-3-[(3S)-3-(acetyloxy)-3-(4-fluorophenyl)propyl]-1-[4-(acetyloxy)phenyl]-4-oxoazetidin-2-yl}-3′-(benzyloxy)biphenyl-3-yl]-6-[(acetyloxy)methyl]tetrahydro-2H-pyran-3,4,5-triyl triacetate

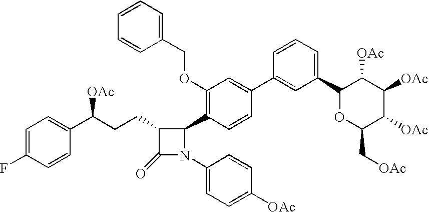

The title compound is prepared from 4-(2S,3R)-3-[(3S)-3-(acetyloxy)-3-(4-fluorophenyl)propyl]-2-[2-(benzyloxy)-4-(4,4,5,5-tetramethyl-1,3,2-dioxaborolan-2-yl)phenyl]-4-oxoazetidin-1-yl}phenyl acetate (i-9) and (2R,3R,4R,5S,6S)-2-[(acetyloxy)methyl]-6-(3-bromophenyl)tetrahydro-2H-pyran-3,4,5-trityl triacetate (i-16) using the procedure described in Example 1, Step A.

### Step B: Preparation of (2S,3S,4R,5R,6R)-2-[4′-[(2S,3R)-3-[(3S)-3-(acetyloxy)-3-(4-fluorophenyl)propyl]-4-oxo-1-(4-{(trifluoromethyl)sulfonyl]oxy}phenyl]azetidin-2-yl]-3′-(benzyloxy)biphenyl-3-yl]-6-[(acetyloxy)methyl]tetrahydro-2H-pyran-3,4,5-triyl triacetate

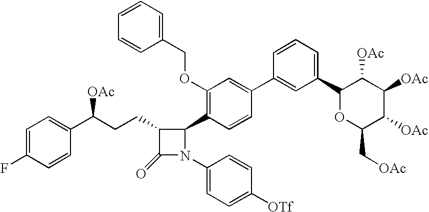

The title compound is prepared from (2S,3S,4R,5R,6R)-2-[4′-{(2S,3R)-3-[(3S)-3-(acetyloxy)-3-(4-fluorophenyl)propyl]1-[4-(acetyloxy)phenyl]-4-oxoazetidin-2-yl}-3′-(benzyloxy)biphenyl-3-yl]-6-[(acetyloxy)methyl]tetrahydro-2H-pyran-3,4,5-triyl triacetate (Example 2, Step A) using the procedure described in Example 1, Step B.

### Step C: Preparation of (2S,3S,4R,5R,6R)-2-[4′-((2S,3R)-3-[(3S)-3-(acetyloxy)-3-(4-fluorophenyl)propyl]-4-oxo-1-{4-[(1-trityl-1H-1,2,4-triazol-3yl)ethynyl]phenyl}azetidin-2-yl]-3′-(benzyloxy)biphenyl-3-yl]-6-[(acetyloxy)methyl]tetrahydro-2H-pyran-3,4,5-triyl triacetate

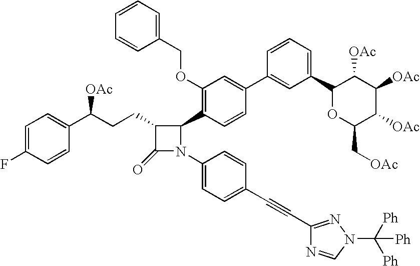

The title compound is prepared from (2S,3S,4R,5R,6R)-2-[4′-[(2S,3R)-3-[(3S)-3-(acetyloxy)-3-(4-fluorophenyl)propyl]-4-oxo-1-(4-{(trifluoromethyl)sulfonyl]oxy}phenyl]azetidin-2-yl]-3′-(benzyloxy)biphenyl-3-yl]-6-[(acetyloxy)methyl]tetrahydro-2H-pyran-3,4,5-triyl triacetate (Example 2, Step B) and 3-ethynyl-1-trityl-1H-1,2,4-triazole (i-30) using the procedure described in Example 1, Step C.

### Step D: Preparation of (3R,4S)-4-{3-(benzyloxy)-3′-[(2S,3R,4R,5S,6R)-3,4,5-trihydroxy-6-(hydroxymethyl)tetrahydro-2H-pyran-2-yl]biphenyl-4-yl}-3-[(3S)-3-(4-fluorophenyl)-3-hydroxypropyl]-1-{4-[(1-trityl-1H-1,2,4-triazol-3-yl)ethynyl]phenyl)azetidin-2-one

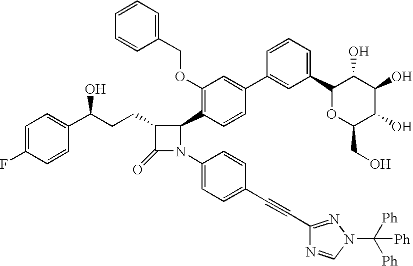

The title compound is prepared from (2S,3S,4R,5R,6R)-2-[4′-((2S,3R)-3-[(3S)-3-(acetyloxy)-3-(4-fluorophenyl)propyl]-4-oxo-1-{4-[(1-trityl-1H-1,2,4-triazol-3-yl)ethynyl]phenyl}azetidin-2-yl]-3′-(benzyloxy)biphenyl-3-yl]-6-[(acetyloxy)methyl]tetrahydro-2H-pyran-3,4,5-triyl triacetate (Example 2, Step C) using the procedure described in Example 1, Step F.

### Step E: Preparation of (3R,4S)-3-[(3S)-3-(4-fluorophenyl)-3-hydroxypropyl]-4-{3-hydroxy-3′-[(2S,3R,4R,5S,6R)-3,4,5-trihydroxy-6-(hydroxymethyl)tetrahydro-2H-pyran-2-yl]biphenyl-4-yl}-1-{4-[2-(1H-1,2,4-triazol-3-yl)ethyl]phenyl)azetidin-2-one

The title compound is prepared from (3R,4S)-4-{3-(benzyloxy)-3′-[(2S,3R,4R,5S,6R)-3,4,5-trihydroxy-6-(hydroxymethyl)tetrahydro-2H-pyran-2-yl]biphenyl-4-yl}-3-[(3S)-3-(4-fluorophenyl)-3-hydroxypropyl]-1-{4-[(1-trityl-1H-1,2,4-triazol-3-yl)ethynyl]phenyl)azetidin-2-one (Example 2, Step D) using the procedure described in Example 1, Step G.

### EXAMPLE 3

### (3R,4S)-3-[(3S)-3-(4-fluorophenyl)-3-hydroxypropyl]-4-{3-hydroxy-3′-[(2S,3R,4R,5S,6R)-3,4,5-trihydroxy-6-(hydroxymethyl)tetrahydro-2H-pyran-2-yl]biphenyl-4-yl}-1-{4-[2-(1,3-thiazol-5-yl)ethyl]phenyl}azetidin-2-one

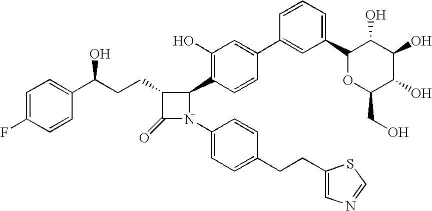

### Step A: Preparation of (2S,3S,4R,5R,6R)-2-[4′-[(2S,3R)-3-[(3S)-3-(acetyloxy)-3-(4-fluorophenyl)propyl]-4-oxo-1-{4-[(trimethylsilyl)ethynyl]phenyl}azetidin-2-yl]-3′-(benzyloxy)biphenyl-3-yl]-6-[(acetyloxy)methyl]tetrahydro-2H-pyran-3,4,5-triyl triacetate

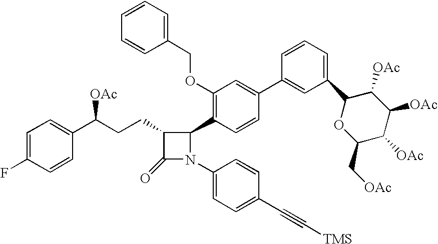

Nitrogen gas is bubbled through a solution of (2S,3S,4R,5R,6R)-2-[4′-[(2S,3R)-3-[(3S)-3-(acetyloxy)-3-(4-fluorophenyl)propyl]-4-oxo-1-(4-{(trifluoromethyl)sulfonyl]oxy}phenyl]azetidin-2-yl]-3′-(benzyloxy)biphenyl-3-yl]-6-[(acetyloxy)methyl]tetrahydro-2H-pyran-3,4,5-triyl triacetate (Example 2, Step B), trimethylsilylacetylene, tetra-n-butylammonium iodide, and triethylamine in anhydrous DMF for a time between 15 and 30 minutes. A suitable palladium catalyst such as tetrakistriphenylphosphine palladium and copper iodide is added, and the resulting reaction mixture is heated between 40° C. and 100° C. under inert atmosphere for a time between 12-24 hr. The reaction mixture is cooled to ambient temperature and poured into water. Using an appropriate solvent, such as ethyl acetate, the product is extracted from the aqueous mixture. The organic extracts are combined and washed with water, brine, and are dried over a suitable drying agent such as Na₂SO₄, filtered and the solvent is removed under vacuum. The residue is purified by MPLC or silica gel flash column chromatography with a gradient eluant from 0% EtOAc/hexanes to 80% EtOAc/hexanes, affording the title compound.

### Step B: Preparation of (2S,3S,4R,5R,6R)-2-[4′-[(2S,3R)-3-[(3S)-3-(acetyloxy)-3-(4-fluorophenyl)propyl]-1-(4-ethynylphenyl)-4-oxoazetidin-2-yl]-3′-(benzyloxy)biphenyl-3-yl]-6-[(acetyloxy)methyl]tetrahydro-2H-pyran-3,4,5-triyl triacetate

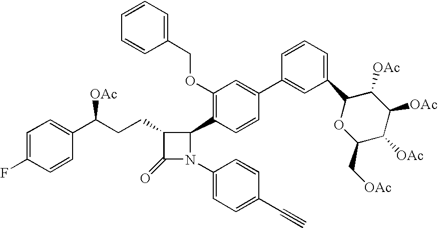

To a solution of (2S,3S,4R,5R,6R)-2-[4′-[(2S,3R)-3-[(3S)-3-(acetyloxy)-3-(4-fluorophenyl)propyl]-4-oxo-1-{4-[(trimethylsilyl)ethynyl]phenyl}azetidin-2-yl]-3′-(benzyloxy)biphenyl-3-yl]-6-[(acetyloxy)methyl]tetrahydro-2H-pyran-3,4,5-triyl triacetate (Example 3, Step A) in a suitable solvent such anhydrous THF or ether that is cooled to 0° C. using an ice bath is added slowly a 1.0M solution of tetra-n-butylammonium fluoride. The resulting reaction mixture is stirred with continued cooling for a period between 0.5 to 2 hr. The reaction mixture is then diluted with water and extracted with the appropriate solvent, such as dichloromethane. The organic layer is dried over sodium or magnesium sulfate, filtered and the solvent is removed under vacuum. The residue is purified by MPLC or silica get flash column chromatography with a gradient eluant from 0% EtOAc/hexanes to 80% EtOAc/hexanes, affording the title compound.

### Step C: Preparation of (2S,3S,4R,5R,6R)-2-[4′-{(2S,3R)-3-[(3S)-3-(acetyloxy)-3-(4-fluorophenyl)propyl]-4-oxo-1-[4-(1,3-thiazol-5-ylethynyl)phenyl]azetidin-2-yl}-3′-(benzyloxy)biphenyl-3-yl]-6-[(acetyloxy)methyl]tetrahydro-2H-pyran-3,4,5-triyl triacetate

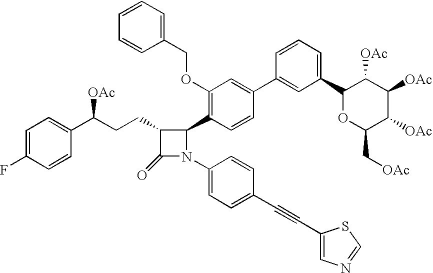

Nitrogen gas is bubbled through a solution of (2S,3S,4R,5R,6R)-2-[4′-[(2S,3R)-3-[(3S)-3-(acetyloxy)-3-(4-fluorophenyl)propyl]-1-(4-ethynylphenyl)-4-oxoazetidin-2-yl]-3′-(benzyloxy)biphenyl-3-yl]-6-[(acetyloxy)methyl]tetrahydro-2H-pyran-3,4,5-triyl triacetate (Example 3, Step B), 5-iodo-1,3-thiazole, triethylamine, and an initiator such as tetra-n-butylammonium iodide in an appropriate solvent such as anhydrous DMF which is then heated between 40° C. and 100° C. for a time between 10-20 minutes. A suitable palladium catalyst such as tetrakistriphenylphosphine palladium and copper iodide is added and the resulting reaction mixture is heated between 40° C. and 100° C. under inert atmosphere for a time between 12-24 hr. The reaction mixture is cooled to ambient temperature and poured into water. Using an appropriate solvent, such as ethyl acetate, the product is extracted from the aqueous mixture. The organic extracts are combined and washed with water, brine, and are dried over a suitable drying agent such as Na₂SO₄, filtered and the solvent is removed under vacuum. The residue is purified by MPLC or silica gel flash column chromatography with a gradient eluant from 0% EtOAc/hexanes to 80% EtOAc/hexanes, affording the title compound.

### Step D: Preparation of (2S,3S,4R,5R,6R)-2-[4′-{(2S,3R)-3-[(3S)-3-(acetyloxy)-3-(4-fluorophenyl)propyl]-4-oxo-1-{4-[2-(1,3-thiazol-5-yl)ethyl]phenyl}azetidin-2-yl}-3′-(benzyloxy)biphenyl-3-yl]-6-[(acetyloxy)methyl]tetrahydro-2H-pyran-3,4,5-triyl triacetate

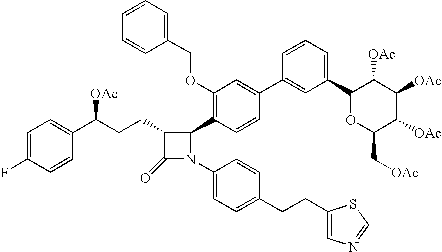

The title compound is prepared from (2S,3S,4R,5R,6R)-2-[4′-{(2S,3R)-3-[(3S)-3-(acetyloxy)-3-(4-fluorophenyl)propyl]-4-oxo-1-[4-(1,3-thiazol-5-ylethynyl)phenyl]azetidin-2-yl}-3′-(benzyloxy)biphenyl-3-yl]-6-[(acetyloxy)methyl]tetrahydro-2H-pyran-3,4,5-triyl triacetate (Example 3, Step C) using the procedure described in Example 1, Step D.

### Step E: Preparation of (3R,4S)-4-{3-(benzyloxy)-3′-[(2S,3R,4R,5S,6R)-3,4,5-trihydroxy-6-(hydroxymethyl)tetrahydro-2H-pyran-2-yl]biphenyl-4-yl}-3-[(3S)-3-(4-fluorophenyl)-3-hydroxypropyl]-1-{4-[2-(1,3-thiazol-5-yl)ethyl]phenyl}azetidin-2-one

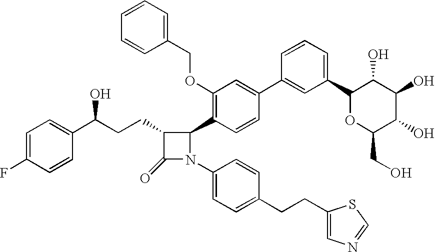

The title compound is prepared (2S,3S,4R,5R,6R)-2-[4′-{(2S,3R)-3-[(3S)-3-(acetyloxy)-3-(4-fluorophenyl)propyl]-4-oxo-1-{4-[2-(1,3-thiazol-5-yl)ethyl]phenyl}azetidin-2-yl}-3′-(benzyloxy)biphenyl-3-yl]-6-[(acetyloxy)methyl]tetrahydro-2H-pyran-3,4,5-triyl triacetate (Example 3, Step D) using the procedure described in Example 1, Step F.

### Step F: Preparation of (3R,4S)-3-[(3S)-3-(4-fluorophenyl)-3-hydroxypropyl]-4-{3-hydroxy-3′-[(2S,3R,4R,5S,6R)-3,4,5-trihydroxy-6-(hydroxymethyl)tetrahydro-2H-pyran-2-yl]biphenyl-4-yl}-1-{4-[2-(,3-thiazol-5-yl)ethyl]phenyl}azetidin-2-one

The title compound is prepared from (3R,4S)-4-{3-(benzyloxy)-3′-[(2S,3R,4R,5S,6R)-3,4,5-trihydroxy-6-(hydroxymethyl)tetrahydro-2H-pyran-2-yl]biphenyl-4-yl}-3-[(3S)-3-(4-fluorophenyl)-3-hydroxypropyl]-1-{4-[2-(1,3-thiazol-5-yl)ethyl]phenyl}azetidin-2-one (Example 3, Step E) using the procedure described in Example 1, Step G.

Using procedures similar to those described above, additional compounds of this invention may be prepared, including but not limited to those described below in Table 1.

TABLE 1
Compounds having the structural formula:

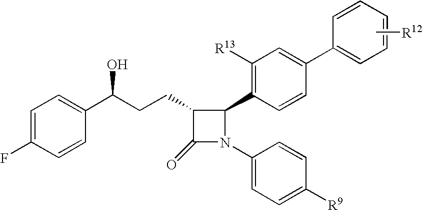

and the pharmaceutically acceptable salts thereof wherein
R¹³ is selected from the group consisting of —H, —F, and —OH;
R¹² is selected from the ortho-, meta-, or para- substituent on the phenyl
ring selected from the group consisting of

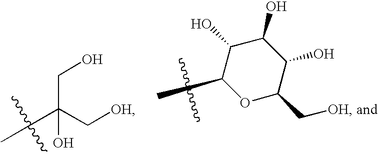

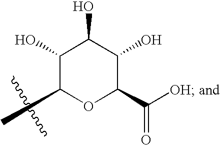

R⁹ is selected from the group consisting of:

| Example # | R⁹ |
|---|---|
| 4 | 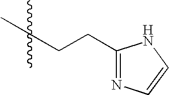 |
| 5 | 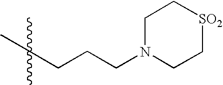 |
| 6 | 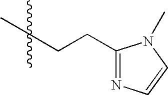 |
| 7 | 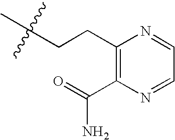 |
| 8 | 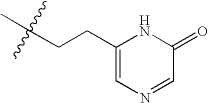 |
| 9 | 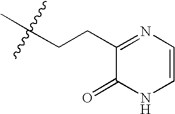 |
| 10 | 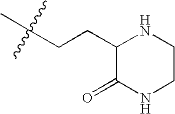 |
| 11 | 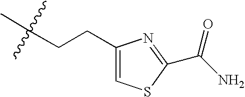 |
| 12 | 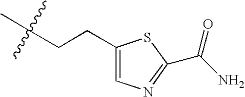 |
| 13 | 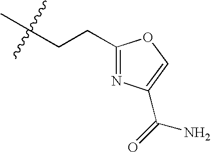 |
| 14 | 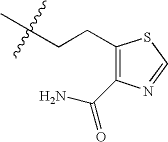 |
| 15 | 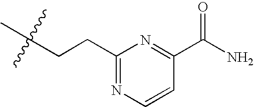 |
| 16 | 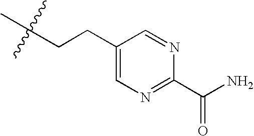 |
| 17 | 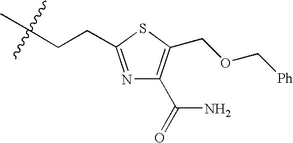 |
| 18 | 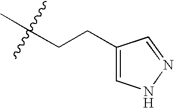 |
| 19 | 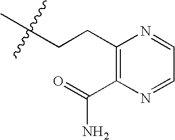 |
| and 20 | 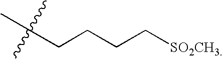 |

### EXAMPLE 21

### (3R,4S)-4-[4-(2-{4-[1,2-dihydroxy-1-(hydroxymethyl)ethyl]phenyl}ethyl)-2-hydroxyphenyl]-3-[(3S)-3-(4-fluorophenyl)-3-hydroxypropyl]-1-{4-[4-(methylsulfonyl)butyl]phenyl}azetidin-2-one

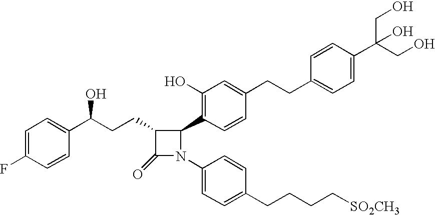

### Step A: Preparation of (1S)-3-[(2S,3R)-2-(2-(benzyloxy)-4-{(E)-2-[4-(5-hydroxy-2,2-dimethyl-1,3-dioxan-5-yl)phenyl]vinyl}phenyl)-4-oxo-1-(4-{[(trifluoromethyl)sulfonyl]oxy}phenyl)azetidin-3-yl]-1-(4-fluorophenyl)propyl acetate

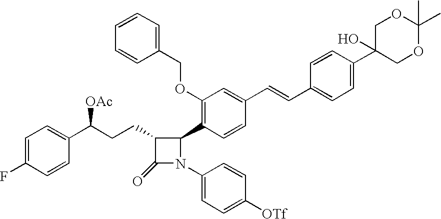

To a solution of (1S)-3-[(2S,3R)-2-[2-(benzyloxy)-4-vinylphenyl]-4-oxo-1-(4-{[(trifluoromethyl)sulfonyl]oxy}phenyl)azetidin-3-yl]-1-(4-fluorophenyl)propyl acetate (which is prepare using procedure for i-32 but replacing allyl-tributyltin with vinyl-tributyltin) and 2,2-dimethyl-5-(4-vinylphenyl)-1,3-dioxan-5-ol (i-33) in a suitable solvent such as anhydrous dichloromethane is added Zhan I or Zhan II catalyst and the resulting mixture is stirred under inert atmosphere at ambient temperature for a time between 16-24 hrs. The solvent is then removed under vacuum and the residue is purified by MPLC or silica gel flash column chromatography with a gradient eluant from 0% EtOAc/hexanes to 80% EtOAc/hexanes to afford the title compound.

### Step B: Preparation of (1S)-3-((2S,3R)-2-(2-(benzyloxy)-4-{(E)-2-[4-(5-hydroxy-2,2-dimethyl-1,3-dioxan-5-yl)phenyl]vinyl}phenyl)-1-{4-[4-{[(methylsulfonyl)but-1-yn-1-yl]phenyl}-4-oxoazetidin-3-yl]-1-(4-fluorophenyl)propyl acetate

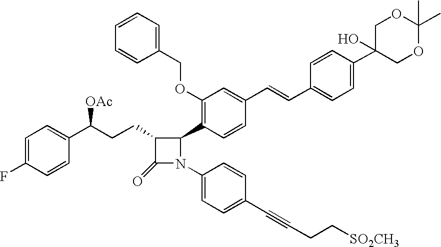

The title compound is prepared from (1S)-3-[(2S,3R)-2-(2-(benzyloxy)-4-{(E)-2-[4-(5-hydroxy-2,2-dimethyl-1,3-dioxan-5-yl)phenyl]vinyl}phenyl)-4-oxo-1-(4-{[(trifluoromethyl)sulfonyl]oxy}phenyl)azetidin-3-yl]-1-(4-fluorophenyl)propyl acetate (Example 21, Step A) and 4-(methylsulfonyl)but-1-yne (i-25) using the procedure described in Example 1, Step C.

### Step C: Preparation of (1S)-3-((2S,3R)-2-(2-(benzyloxy)-4-{2-[4-(5-hydroxy-2,2-dimethyl-1,3-dioxan-5-yl)phenyl]ethyl}phenyl)-1-{4-[4-{[(methylsulfonyl)butyl]phenyl}-4-oxoazetidin-3-yl]-1-(4-fluorophenyl)propyl acetate

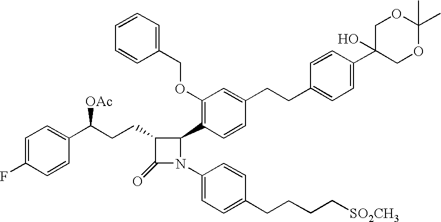

The title compound is prepared from (1S)-3-((2S,3R)-2-(2-(benzyloxy)-4-{(E)-2-[4-(5-hydroxy-2,2-dimethyl-1,3-dioxan-5-yl)phenyl]vinyl}phenyl)-1-{4-[4-{[(methylsulfonyl)but-1-yn-1-yl]phenyl}-4-oxoazetidin-3-yl]-1-(4-fluorophenyl)propyl acetate (Example 21, Step B) using the procedure described in Example 1, Step D.

### Step D: Preparation of (1S)-3-((2S,3R)-2-(2-(benzyloxy)-4-(2-{4-[1,2-dihydroxy-1-(hydroxymethyl)ethyl]phenyl}ethyl)phenyl]-1-{4-[4-(methylsulfonyl)butyl]phenyl}-4-oxoazetidin-3-yl]-1-(4-fluorophenyl)propyl acetate

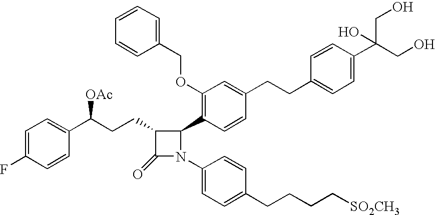

The title compound is prepared from (1S)-3-((2S,3R)-2-(2-(benzyloxy)-4-{2-[4-(5-hydroxy-2,2-dimethyl-1,3-dioxan-5-yl)phenyl]ethyl}phenyl)-1-{4-[4-{[(methylsulfonyl)butyl]phenyl}-4-oxoazetidin-3-yl]-1-(4-fluorophenyl)propyl acetate (Example 21, Step C) using the procedure described in Example 1, Step E.

### Step E: Preparation of (3R,4S)-4-[2-(benzyloxy)-4-(2-{4-[1,2-dihydrox-1-(hydrooxymethyl)ethyl]phenyl}ethyl)phenyl]-3-[(3S)-3-(4-fluorophenyl)-3-hydroxypropyl]-1-{4-[4-(methylsulfonyl)butyl]phenyl}azetidin-2-one

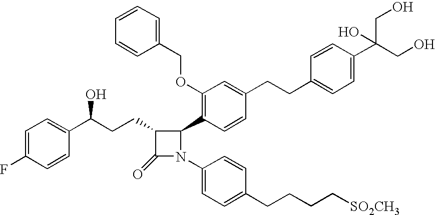

The title compound is prepared (1S)-3-((2S,3R)-2-(2-(benzyloxy)-4-(2-{4-[1,2-dihydroxy-1-(hydroxymethyl)ethyl]phenyl}ethyl)phenyl]-1-{4-[4-(methylsulfonyl)butyl]phenyl}-4-oxoazetidin-3-yl]-1-(4-fluorophenyl)propyl acetate (Example 21, Step D) using the procedure described in Example 1, Step F.

### Step F: Preparation of (3R,4S)-4-[4-(2-{4-[1,2-dihydroxy-1-(hydroxymethyl)ethyl]phenyl}ethyl)-2-hydroxyphenyl]-3-[(3S)-3-(4-fluorophenyl)-3-hydroxypropyl]-1-{4-[4-(methylsulfonyl)butyl]phenyl}azetidin-2-one

The title compound is prepared from (3R,4S)-4-[2-(benzyloxy)-4-(2-{4-[1,2-dihydrox-1-(hydrooxymethyl)ethyl]phenyl}ethyl)phenyl]-3-[(3S)-3-(4-fluorophenyl)-3-hydroxypropyl]-1-{4-[4-(methylsulfonyl)butyl]phenyl}azetidin-2-one (Example 21, Step E) using the procedure described in Example 1, Step G.

### EXAMPLE 22

### (3R,4S)-3-[(3S)-3-(4-fluorophenyl)-3-hydroxypropyl]-4-(4-{3-[2-(6-hydroxyhexyl)-phenyl]propyl}phenyl)-1-{4-[3-(1H-1,2,3-triazol-1-yl)propyl]phenyl}azetidin-2-one

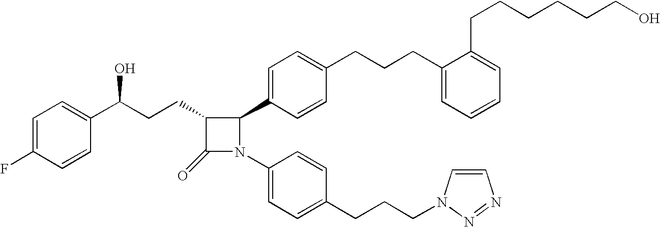

### Step A: Preparation of (1S)-1-(4-fluorophenyl)-3-[(2S,3R)-2-{4-[(2E)-3-(2-iodophenyl)prop-2-en-1-yl]phenyl}-4-oxo-1-(4-{[(trifluoromethyl)sulfonyl]oxy}phenyl)azetidin-3-yl]propyl acetate

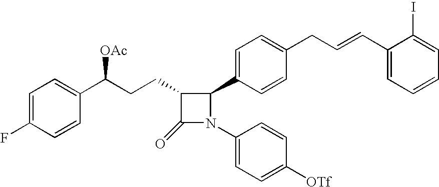

The title compound is prepared from (1S)-3-[(2S,3R)-2-[4-allyl-2-(benzyloxy)phenyl]-4-oxo-1-(4-{[(trifluoromethyl)sulfonyl]oxy}phenyl)azetidin-3-yl]-1-(4-fluorophenyl)propyl acetate (i-32) and o-iodostyrene using the procedure described in Example 21, Step A.

### Step B: Preparation of (1S-3-[(2S,3R)-2-[4-((2E)-3-{2-[6-(benzyloxy)hex-1-yn-1′-yl]phenyl}prop-2-en-1-yl)phenyl]-4-oxo-1-(4-{[(trifluoromethyl)sulfonyl]oxy}phenyl)azetidin-3-yl]-1-(4-fluorophenyl)propyl acetate

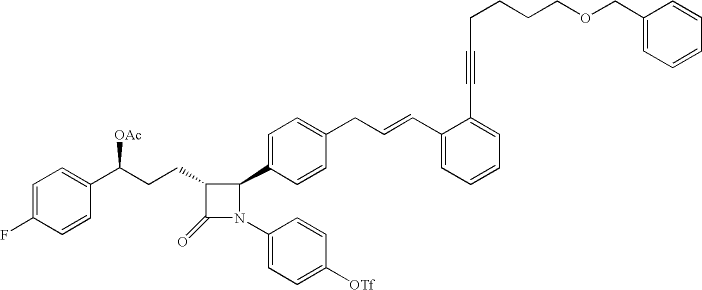

To a solution of (1S)-1-(4-fluorophenyl)-3-[(2S,3R)-2-{4-[(2E)-3-(2-iodophenyl)prop-2-en-1-yl]phenyl}-4-oxo-1-(4-{[(trifluoromethyl)sulfonyl]oxy}phenyl)azetidin-3-yl]propyl acetate (Example 22, Step A), copper iodide, triethylamine, and benzyl hex-5-yn-1-yl ether (i-24) in a suitable solvent such as anhydrous DMF is added the appropriate palladium catalyst, such as dichloro-bis(triphenylphospine)palladium or tetrakistriphenylphosphine palladium, and the resulting mixture is set under inert atmosphere and stirred at ambient temperature for a period of 6-24 hrs. The reaction mixture is poured into 0.5 to 2N aqueous hydrochloric acid, and, using an appropriate solvent such as ethyl acetate, the product is extracted from the aqueous mixture. The organic extracts are combined and washed with water, brine, and dried over a suitable drying agent such as Na₂SO₄ and filtered, and the solvent is removed under vacuum. The residue is purified by MPLC or silica gel flash column chromatography with a gradient eluant from 0% EtOAc/hexanes to 80% EtOAc/hexanes to afford the title compound.

### Step C: Preparation of (1S)-3-((2S,3R)-2-[4-((2E)-3-{2-[6-(benzyloxy)hex-1-yn-1-yl]phenyl}prop-2-en-1-yl)phenyl]-4-oxo-1-{4-[3-(1H-1,2,3-triazol-1-yl)prop-1-yn-1-yl]phenyl}azetidin-3-yl]-1-(4-fluorophenyl)propyl acetate

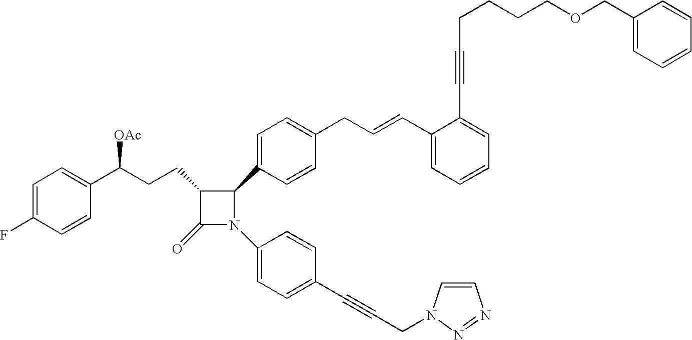

The title compound may be prepared from (1S)-3-[(2S,3R)-2-[4-((2E)-3-{2-[6-(benzyloxy)hex-1-yn-1-yl]phenyl}prop-2-en-1-yl)phenyl]-4-oxo-1-(4-{[(trifluoromethyl)sulfonyl]oxy}phenyl)azetidin-3-yl]-1-(4-fluorophenyl)propyl acetate (Example 22, Step B) and 1-prop-2-yn-1-yl-1H-1,2,4-triazole (i-27) using the procedure described in Example 1, Step C.

### Step D: Preparation of (1S)-1-(4-fluorophenyl)-3-((2S,3R)-2-(4-{3-[2-(6-hydroxyhexyl)phenyl]propyl}phenyl)-4-oxo-1-{4-[3-(1H-1,2,3-triazol-1-yl)propyl]phenyl}azetidin-3-yl)propyl acetate

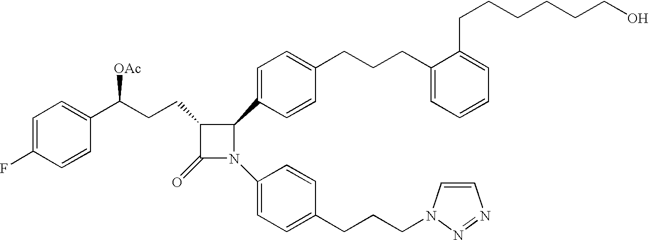

The title compound is prepared from (1S)-3-((2S,3R)-2-[4-((2E)-3-{2-[6-(benzyloxy)hex-1-yn-1-yl]phenyl}prop-2-en-1-yl)phenyl]-4-oxo-1-{4-[3-(1H-1,2,3-triazol-1-yl)prop-1-yn-1-yl]phenyl}azetidin-3-yl]-1-(4-fluorophenyl)propyl acetate (Example 22, Step C) using the procedure described in Example 1, Step G.

### Step E: Preparation of (3R,4S)-3-[(3S)-3-(4-fluorophenyl)-3-hydroxypropyl]-4-(4-{3-[2-(6-hydroxyhexyl)phenyl]propyl}phenyl)-1-{4-[3-(1H-1,2,3-triazol-1-yl)propyl]phenyl}azetidin-2-one

The title compound is prepared (1S)-1-(4-fluorophenyl)-3-((2S,3R)-2-(4-{3-[2-(6-hydroxyhexyl)phenyl]propyl}phenyl)-4-oxo-1-{4-[3-(1H-1,2,3-triazol-1-yl)propyl]phenyl}azetidin-3-yl)propyl acetate (Example 22, Step D) using the procedure described in Example 1, Step F.

Using procedures similar to those described above, additional compounds of this invention may be prepared, including but not limited to those described below in Table 2.

TABLE 2
Compounds having the structural formula:

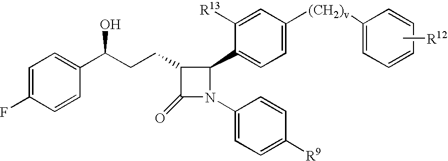

and the pharmaceutically acceptable salts thereof, wherein:
R¹³ is selected from the group consisting of —H, —F, and —OH;
R¹² is —C₁₋₈alkyl mono- or poly-substituted with —OH or —COOH,
or both —OH and —COOH; and v and R⁹ are defined as follows:

| Example # | v | R⁹ |
|---|---|---|
| 23 | 2 | —(CH₂)₄—SO₂CH₃ |
| 24 | 2 | —(CH₂)₅—SO₂CH₃ |
| 25 | 2 | —(CH₂)₆—SO₂CH₃ |
| 26 | 3 | —(CH₂)₄—SO₂CH₃ |
| 27 | 3 | —(CH₂)₅—SO₂CH₃ |
| 28 | 3 | —(CH₂)₆—SO₂CH₃ |
| 29 | 4 | —(CH₂)₄—SO₂CH₃ |
| 30 | 4 | —(CH₂)₅—SO₂CH₃ |
| 31 | 4 | —(CH₂)₆—SO₂CH₃ |
| 32 | 5 | —(CH₂)₄—SO₂CH₃ |
| 33 | 5 | —(CH₂)₅—SO₂CH₃ |
| 34 | 5 | —(CH₂)₆—SO₂CH₃ |
| 35 | 6 | —(CH₂)₄—SO₂CH₃ |
| 36 | 2 | 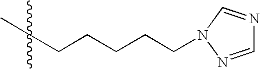 |
| 37 | 3 | 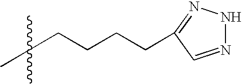 |
| 38 | 4 | 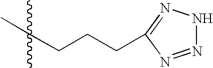 |
| 39 | 5 | 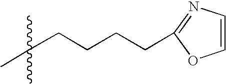 |
| 40 | 6 | 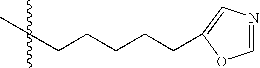 |
| 41 | 2 | 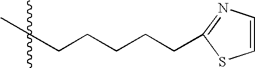 |
| 42 | 3 | 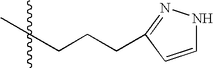 |
| 43 | 4 | 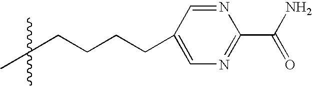 |
| 44 | 5 | 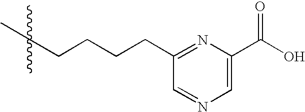 |
| 45 | 6 | 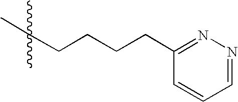 |
| 46 | 2 | 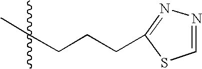 |
| 47 | 3 | 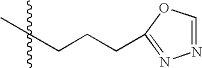 |
| 48 | 4 | —(CH₂)₃—NHSO₂CH₃ |
| 49 | 5 | —(CH₂)₄—NHSO₂CH₃ |
| and 50 | 6 | —(CH₂)₅—NHSO₂CH₃ |

### EXAMPLE 51

### (3-[4′-((2S,3R)-3-[(3S)-3-(4-fluorophenyl)-3-hydroxypropyl]-1-{4-[2-(1-methyl-1H-imidazol-2-yl)ethyl]phenyl}-4-oxoazetidin-2-yl)-3′-hydroxybiphenyl-4-yl]propyl}malonic acid

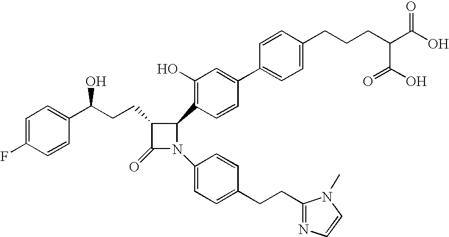

### Step A: Preparation of 4-{(2S,3R)-3-[(3S)-3-(acetyloxy)-3-(4-fluorophenyl)propyl]-2-[3-(benzyloxy)-4′-bromobiphenyl-4-yl]-4-oxoazetidin-1-yl}phenyl acetate

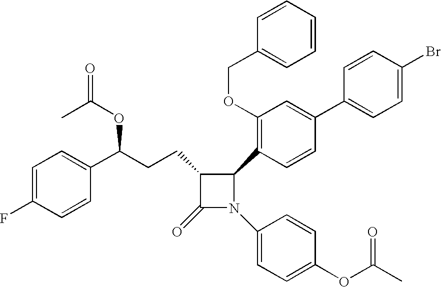

The title compound is prepared from 4-(2S,3R)-3-[(3S)-3-(acetyloxy)-3-(4-fluorophenyl)propyl]-2-[2-(benzyloxy)-4-(4,4,5,5-tetramethyl-1,3,2-dioxaborolan-2-yl)phenyl]-4-oxoazetidin-1-yl}phenyl acetate (i-9) and 1,4-dibromobenzene using the procedure described in Example 1, Step A.

### Step B: Dibenzyl {3-[4′-{(2S,3R)-3-[(3S)-3-(acetyloxy)-3-(4-fluorophenyl)propyl]-1-[4-(acetyloxy)phenyl]-4-oxoazetidin-2-yl}-3′-(benzyloxy)biphenyl-4-yl]prop-2-yn-1-yl}malonate

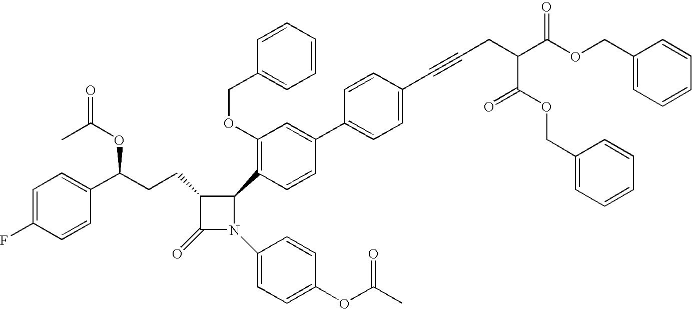

To a solution of 4-{(2S,3R)-3-[(3S)-3-(acetyloxy)-3-(4-fluorophenyl)propyl]-2-[3-(benzyloxy)-4′-bromobiphenyl-4-yl]-4-oxoazetidin-1-yl}phenyl acetate (Example 51, Step A), copper iodide, triethylamine, and dibenzyl ethynylmalonate (i-26) in a suitable solvent such as anhydrous DMF is added the appropriate palladium catalyst, such as dichloro-bis(triphenylphospine)palladium or tetrakistriphenylphosphine palladium, and the resulting mixture is set under inert atmosphere and heated to a temperature between room temperature to 70° C. for a period of 6-24 hrs. The reaction mixture is poured into 0.5 to 2N aqueous hydrochloric acid, and using an appropriate solvent, such as ethyl acetate, the product is extracted from the aqueous mixture. The organic extracts are combined and washed with water, brine, dried over a suitable drying agent such as Na₂SO₄, and filtered, and the solvent is removed under vacuum. The residue is purified by MPLC or silica gel flash column chromatography with a gradient eluant from 0% EtOAc/hexanes to 80% EtOAc/hexanes to afford the title compound.

### Step C: Dibenzyl {3-[4′-[(2S,3R)-3-[(3S)-3-(acetyloxy)-3-(4-fluorophenyl)propyl]-4-oxo-1-(4-{[(trifluoromethyl)sulfonyl]oxy}phenyl)-azetidin-2-yl}-3′-(benzyloxy)biphenyl-4-yl]prop-2-yn-1-yl}malonate

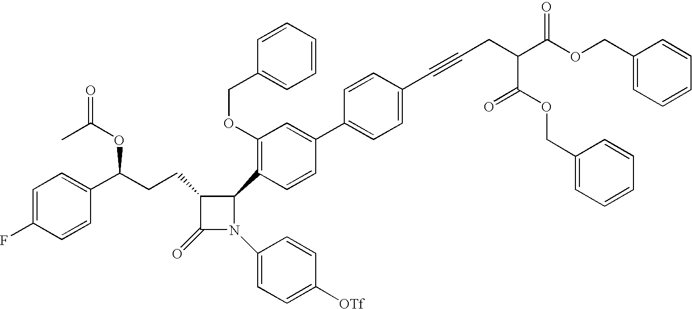

The title compound is prepared from Dibenzyl {3-[4′-{(2S,3R)-3-[(3S)-3-(acetyloxy)-3-(4-fluorophenyl)propyl]-1-[4-(acetoyloxy)phenyl]-4-oxoazetidin-2-yl}-3′-(benzyloxy)biphenyl-4-yl]prop-2-yn-1-yl}malonate (Example 51, Step B) using the procedure described in Example 1, Step B.

### Step D: Dibenzyl {3-[4′-[(2S,3R)-3-[(3S)-3-(acetyloxy)-3-(4-fluorophenyl)propyl]-1-{4-[(1-methyl-1H-imidazol-5-yl)ethynyl]phenyl}-4-oxoazetidin-2-yl}-3′-(benzyloxy)biphenyl-4-yl]prop-2-yn-1-yl}malonate

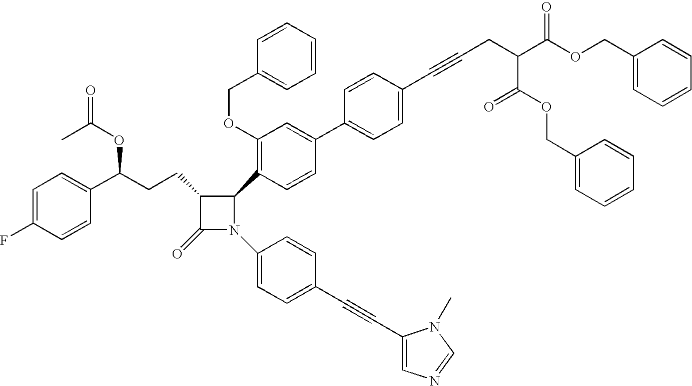

The title compound is prepared from Dibenzyl {3-[4′-[(2S,3R)-3-[(3S)-3-(acetyloxy)-3-(4-fluorophenyl)propyl]-4-oxo-1-(4-{[(trifluoromethyl)sulfonyl]oxy}phenyl)-azetidin-2-yl}-3′-(benzyloxy)biphenyl-4-yl]prop-2-yn-1-yl}malonate (Example 51, Step C) and commercially available 5-ethynyl-1-methyl-1H-imidazole using the procedure described in Example 1, Step C.

### Step E: Dibenzyl {3-[3′-(benzyloxy)-4′-((2S,3R)-3-[(3S)-3-(4-fluorophenyl)-3-hydroxypropyl]-1-{4-[(1-methyl-1H-imidazol-5-yl)ethynyl]phenyl}-4-oxoazetidin-2-yl)biphenyl-4-yl]prop-2-yn-1-yl}-malonate

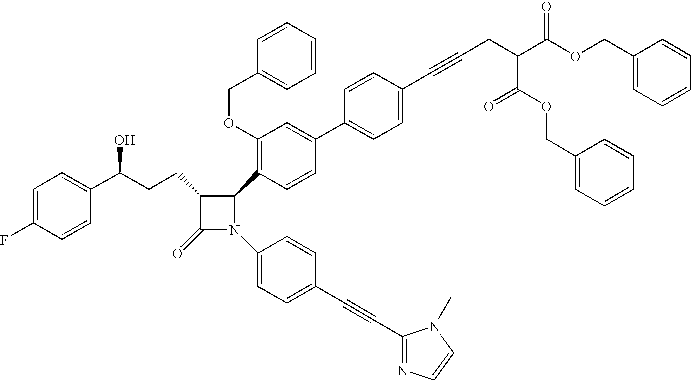

The title compound is prepared from Dibenzyl {3-[4′-[(2S,3R)-3-[(3S)-3-(acetyloxy)-3-(4-fluorophenyl)propyl]-1-{4-[(1-methyl-1H-imidazol-5-yl)ethynyl]phenyl}-4-oxoazetidin-2-yl}-3′-(benzyloxy)biphenyl-4-yl]prop-2-yn-1-yl}malonate (Example 51, Step D) using the procedure described in Example 1, Step F.

### Step F: Preparation of (3-[4′-((2S,3R)-3-[(3S)-3-(4-fluorophenyl)-3-hydroxypropyl]-1-{4-[2-(1-methyl-1H-imidazol-2-yl)ethyl]phenyl}-4-oxoazetidin-2-yl)-3′-hydroxybiphenyl-4-yl]propyl}malonic acid

The title compound is prepared Dibenzyl {3-[3′-(benzyloxy)-4′-((2S,3R)-3-[(3S)-3-(4-fluorophenyl)-3-hydroxypropyl]-1-{4-[(1-methyl-1H-imidazol-5-yl)ethynyl]phenyl}-4-oxoazetidin-2-yl)biphenyl-4-yl]prop-2-yn-1-yl}-malonate (Example 51, Step E) using the procedure described in Example 1, Step G.

Using procedures similar to those described above, additional compounds of this invention may be prepared, including but not limited to those described below in Table 3.

TABLE 3
Compounds having the structural formula:

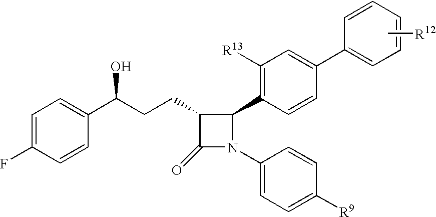

and the pharmaceutically acceptable salts thereof wherein
R¹³ is selected from the group consisting of —H, —F, and —OH; and
R¹² and R⁹ are defined as follows:

| Example # | R¹² | R⁹ |
|---|---|---|
| 52 | 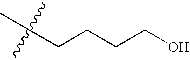 | 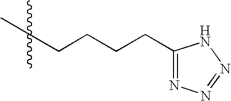 |
| 53 | 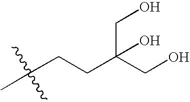 | 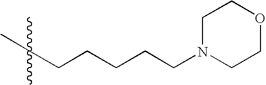 |
| 54 | 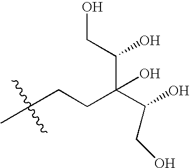 | 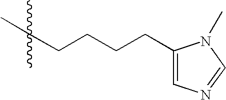 |
| 55 | 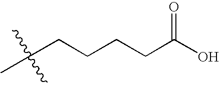 | 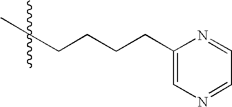 |
| 56 | 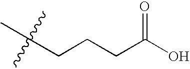 | 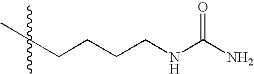 |
| 57 | 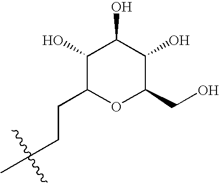 | 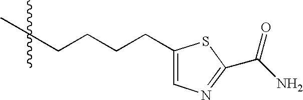 |
| and 58 | 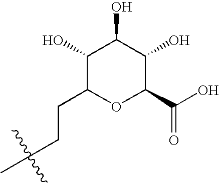 | 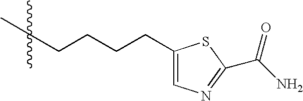 |

While the invention has been described and illustrated with reference to certain particular embodiments thereof, those skilled in the art will appreciate that various changes, modifications and substitutions which may be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. Recitation of a specific compound in the claims (i.e., a species) without a chiral designation is intended to encompass the racemate, racemic mixtures, each individual enantiomer, a diastereoisomeric mixture and each individual diastereoisomer of the compound where such forms are possible due to the presence of one or more asymmetric centers. All patents, patent applications and publications cited herein are incorporated by reference in their entirety. It is intended, therefore, that the invention be defined by the scope of the claims which follow and that such claims be interpreted as broadly as is reasonable.

What is claimed is:

1. A compound of structural Formula I 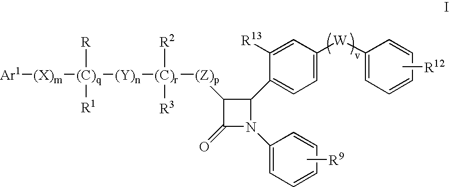
or a pharmaceutically acceptable salt thereof, wherein:
Ar¹ is selected from the group consisting of aryl and R⁴-substituted aryl;
X, Y and Z are independently selected at each occurrence from the group consisting of —CH₂—, —CH(C₁₋₆alkyl)- and —C(C₁₋₆alkyl)₂-;
W is independently selected at each occurrence from the group consisting of —CH₂—, —CH(C₁₋₆alkyl)- and —C(C₁₋₆alkyl)₂-;
v is an integer selected from 0, 1, 2, 3, 4, 5 and 6;
R is selected from the group consisting of —OR⁶, —O(CO)R⁶, —O(CO)OR⁸, —O(CO)NR⁶R⁷, a sugar residue, a disugar residue, a trisugar residue and a tetrasugar residue;
R¹ is selected from the group consisting of —H, —C₁₋₆alkyl and aryl, or R and R¹ together are oxo;
R² is selected from the group consisting of —OR⁶, —O(CO)R⁶, —O(CO)OR⁸ and —O(CO)NR⁶R⁷;
R³ is selected from the group consisting of —H, —C₁₋₆alkyl and aryl, or R² and R³ together are oxo;
q and r are integers each independently selected from 0 and 1 provided that at least one of q and r is 1;
m, n, and p are integers each independently selected from 0, 1, 2, 3 and 4, provided that the sum of m, n, p, q, and r is 1, 2, 3, 4, 5 or 6;
t is an integer selected from 0, 1 and 2;
R⁴ is 1-5 substituents independently selected at each occurrence from the group consisting of: —OR⁵, —O(CO)R⁵, —O(CO)OR⁸, —O—C₁₋₅ alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀alkyl-COOR⁵, —O—C₁₋₁₀alkyl-CONR⁵R⁶, and fluoro;
R⁵, R⁶ and R⁷ are independently selected at each occurrence from the group consisting of —H, —C₁₋₆alkyl, aryl and aryl-substituted —C₁₋₆alkyl;
R⁸ is selected from the group consisting of —C₁₋₆alkyl, aryl and aryl-substituted-C₁₋₆alkyl;
R⁹ is selected from the group consisting of —C₁₋₈alkyl-Hetcy, —(CH₂)₀₋₂CH═CH—C₀₋₆alkyl-Hetcy, —C═C—C₀₋₆alkyl-Hetcy, —C₁₋₈alkyl-NH-Hetcy, —C₁₋₈alkyl-NR¹⁰R¹¹, —(CH₂)₀₋₂CH═CH—C₁₋₆alkyl-NR¹⁰R¹¹, —C≡C—C₁₋₆alkyl-NR¹⁰R¹¹, and —C₁₋₈alkyl-SO₂R⁸;
Hetcy is selected from the group consisting of:
(a) a 5-membered aromatic or partially unsaturated heterocyclic ring containing 1 to 4 heteroatoms selected from 1 to 4 of N, zero to 1 of S, and zero to 1 of O, wherein the heterocyclic ring is optionally mono- or di-substituted with R¹⁴,
(b) a 6-membered aromatic heterocyclic ring containing 1 to 3 N heteroatoms, wherein the heterocyclic ring is optionally mono- or di-substituted with R¹⁴, and
(c) a 6-membered saturated heterocyclic ring containing 1 to 3 heteroatoms selected from 1-3 of N, zero to 1 of O, and zero to 1 of S(O)ₜ, and wherein the heterocyclic ring is optionally mono- or di-substituted with R¹⁴;
R^{10a} is —C1-3alkyl optionally substituted with one or more substituents selected form the group consisting of —OH, 1-3 of fluoro and phenyl;
R¹⁰ is independently selected at each occurrence from the group consisting of —H and —C₁₋₃alkyl optionally substituted with one or more substituents selected form the group consisting of —OH, 1-3 of fluoro and phenyl;
R¹¹ is selected from the group consisting of —H, —C(O)—C₁₋₃alkyl, —C(O)—NR¹⁰R¹⁰, —SO₂—C₁₋₃alkyl, —SO₂-phenyl and —C₁₋₃alkyl optionally substituted with one or more substituents selected form the group consisting of —OH, 1-3 of fluoro and phenyl;
R¹² is selected from the group consisting of (a) —C₁₋₁₅alkyl mono- or poly-substituted with one or more substituents selected from the group consisting of —OH and —COOH, (b) —C₂₋₁₅alkenyl mono- or poly-substituted with one or more substituents selected from the group consisting of —OH and —COOH, (c) —C₂₋₁₅alkynyl mono- or poly-substituted with one or more substituents selected from the group consisting of —OH and —COOH, (d) —C₁₋₃alkyl-C₃₋₆cycloalkyl wherein each carbon in the cycloalkyl ring is optionally substituted with —OH or —COOH, (e) a sugar residue, and (f) —C₁₋₃alkyl substituted with a sugar residue;
R¹³ is selected from the group consisting of —H, —F, and —OH; and
R¹⁴ is independently selected at each occurrence from the group consisting of: —R^{10a}, —C₁₋₃alkyl-COOR¹⁰, —C₁₋₃alkyl-C(O)NR¹⁰R¹⁰, —C₁₋₃alkyl-SO₂—R^{10a}, —C₁₋₃alkyl-O—R^{10a}, —COOR¹⁰, —OC(O)—R^{10a}, —C(O)NR¹⁰R¹⁰, —NR¹⁰R¹⁰, oxo, and hydroxy.

2. The compound of claim 1 having the structural formula Ia 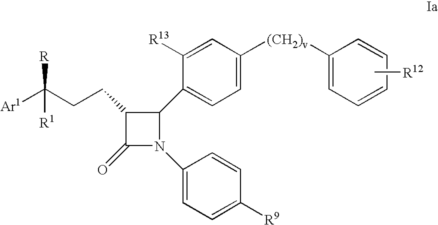
or a pharmaceutically acceptable salt thereof.

3. The compound of claim 2 wherein (a) v is zero or (b) v is selected from 1, 2, 3, 4, 5 and 6 and W is —CH₂—.

4. The compound of claim 2 wherein Ar¹ is selected from the group consisting of phenyl and R⁴-substituted phenyl wherein R⁴ is 1-2 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀alkyl-COOR⁵, —O—C₁₋₁₀alkyl-CONR⁵R⁶ and fluoro.

5. The compound of claim 4 wherein R¹² is selected from the group consisting of (a) —C₁₋₈alkyl mono- or poly-substituted with one or more substituents selected from the group consisting of —OH and —COOH, (b) —C₂₋₈alkenyl mono- or poly-substituted with one or more substituents selected from the group consisting of —OH and —COOH, (c) —C₂₋₈alkynyl mono- or poly-substituted with one or more substituents selected from the group consisting of —OH and —COOH; (d) —C₁₋₃alkyl-C₃₋₆cycloalkyl wherein each carbon in the cycloalkyl ring is optionally substituted with —OH or —COOH, (e) a sugar residue, and (f) —C₁₋₃alkyl substituted with a sugar residue.

6. The compound of claim 5 wherein R⁹ is selected from the group consisting of —C₁₋₈alkyl-Hetcy, —(CH₂)₀₋₂CH═CH—C₀₋₆alkyl-Hetcy, —C≡C—C₀₋₆alkyl-Hetcy, —C₁₋₈alkyl-NH-Hetcy, and —C₁₋₈alkyl-SO₂R⁸.

7. The compound of claim 6 wherein R is —OH and R¹ is —H.

8. The compound of claim 5 wherein R⁹ is selected from the group consisting of —C₁₋₁₈alkyl-NR¹⁰R¹¹, —(CH₂)₀₋₂CH═CH—C₁₋₆alkyl-NR¹⁰OR¹¹, and —C≡C—C₁₋₆alkyl-NR¹⁰OR¹¹.

9. The compound of claim 8 wherein R is —OH and R¹ is —H.

10. The compound of claim 1 having the structural formula Ib 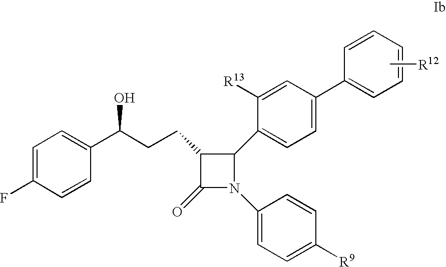
or a pharmaceutically acceptable salt thereof.

11. The compound of claim 10 wherein R¹² is selected from the group consisting of (a) —C₁₋₈alkyl mono- or poly-substituted with one or more substituents selected from the group consisting of —OH and —COOH, (b) —C₂₋₈alkenyl mono- or poly-substituted with one or more substituents selected from the group consisting of —OH and —COOH, (c) —C₂₋₈alkynyl mono- or poly-substituted with one or more substituents selected from the group consisting of —OH and —COOH; (d) —C₁₋₃alkyl-C₃₋₆cycloalkyl wherein each carbon in the cycloalkyl ring is optionally substituted with —OH or —COOH, (e) a sugar residue, and (f) —C₁₋₃alkyl substituted with a sugar residue.

12. The compound of claim 11 wherein R⁹ is selected from the group consisting of —C₁₋₈alkyl-Hetcy, —(CH₂)₀₋₂CH═CH—C₀₋₆alkyl-Hetcy, —C≡C—C₀₋₆alkyl-Hetcy, —C₁₋₈alkyl-NH-Hetcy, and —C₁₋₈alkyl-SO₂R⁸.

13. The compound of claim 11 wherein R⁹ is selected from the group consisting of —C₁₋₈alkyl-NR¹⁰R¹¹, —(CH₂)₀₋₂CH═CH—C₁₋₆alkyl-NR OR¹¹, and —C≡C—C₁₋₆alkyl-NR¹⁰R¹¹.

14. The compound of claim 1 selected from the group consisting of:
N-[3-(4-{(2S,3R)-2-{4′-[1,2-dihydroxy-1-(hydroxymethyl)ethyl]-3-hydroxybiphenyl-4-yl}-3-[(3S)-3-(4-fluorophenyl)-3-hydroxypropyl]-4-oxoazetidin-1-yl}phenyl)propyl]-methanesulfonamide;
(3R,4S)-3-[(3S)-3-(4-fluorophenyl)-3-hydroxypropyl]-4-{3-hydroxy-3′-[(2S,3R,4R,5S,6R)-3,4,5-trihydroxy-6-(hydroxymethyl)tetrahydro-2H-pyran-2-yl]biphenyl-4-yl}-1-{4-[2-(1H-1,2,4-triazol-3-yl)ethyl]phenyl)azetidin-2-one;
(3R,4S)-3-[(3S)-3-(4-fluorophenyl)-3-hydroxypropyl]-4-{3-hydroxy-3′-[(2S,3R,4R,5S,6R)-3,4,5-trihydroxy-6-(hydroxymethyl)tetrahydro-2H-pyran-2-yl]biphenyl-4-yl}-1-{4-[2-(1,3-thiazol-5-yl)ethyl]phenyl}azetidin-2-one;
(3R,4S)-4-[4-(2-{4-[1,2-dihydrox-1-(hydroxymethyl)ethyl]phenyl}ethyl)-2-hydroxyphenyl]-3-[(3S)-3-(4-fluorophenyl)-3-hydroxypropyl]-1-{4-[4-(methylsulfonyl)butyl]phenyl}azetidin-2-one;
(3R,4S)-3-[(3S)-3-(4-fluorophenyl)-3-hydroxypropyl]-4-(4-{3-[2-(6-hydroxyhexyl)-phenyl]propyl}phenyl)-1-{4-[3-(1H-1,2,3-triazol-1-yl)propyl]phenyl}azetidin-2-one;
(3-[4′-((2S,3R)-3-[(3S)-3-(4-fluorophenyl)-3-hydroxypropyl]1-{4-[2-(1-methyl-1H-imidazol-2-yl)ethyl]phenyl}-4-oxoazetidin-2-yl)-3′-hydroxybiphenyl-4-yl]propyl}malonic acid;
and the pharmaceutically acceptable salts thereof.

15. The compound of claim 1 having the structural formula: 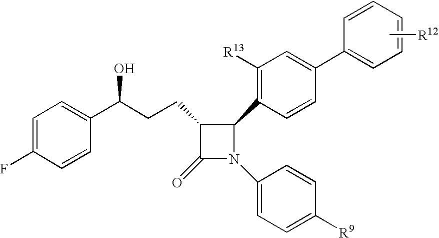
or a pharmaceutically acceptable salt thereof, wherein:
R¹³ is selected from the group consisting of —H, —F, and —OH;
R¹² is selected from the ortho-, meta-, or para-substituent on the phenyl ring selected from the group consisting of 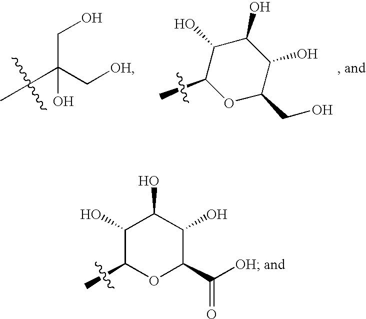
R⁹ is selected from the group consisting of:

| Compound No. | R⁹ |
|---|---|
| 4 | 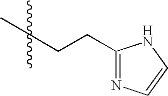 |
| 5 | 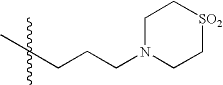 |
| 6 | 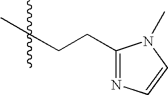 |
| 7 | 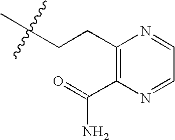 |
| 8 | 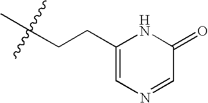 |
| 9 | 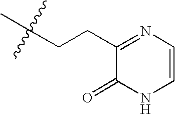 |
| 10 | 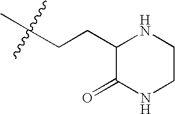 |
| 11 | 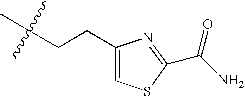 |
| 12 | 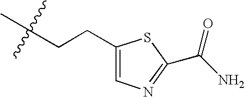 |
| 13 | 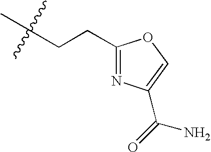 |
| 14 | 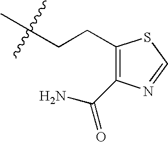 |
| 15 | 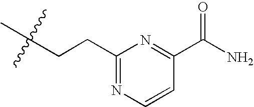 |
| 16 | 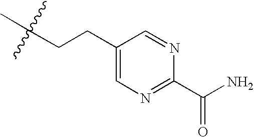 |
| 17 | 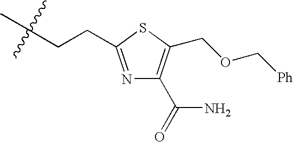 |
| 18 | 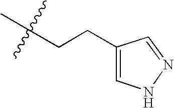 |
| 19 | 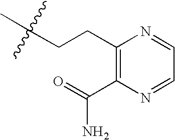 |
| and 20 | 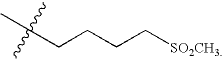 |

16. The compound of claim 1 having the structural formula: 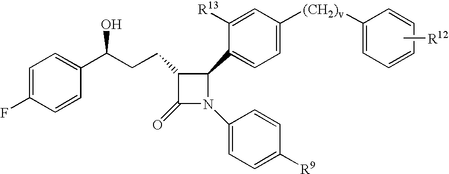
or a pharmaceutically acceptable salt thereof, wherein:
R¹³ is selected from the group consisting of —H, —F, and —OH;
R¹² is —C₁₋₈alkyl mono- or poly-substituted with —OH or —COOH, or both —OH and —COOH; and v and R⁹ are defined as follows:

| Compound No. | v | R⁹ |
|---|---|---|
| 23 | 2 | —(CH₂)₄—SO₂CH₃ |
| 24 | 2 | —(CH₂)₅—SO₂CH₃ |
| 25 | 2 | —(CH₂)₆—SO₂CH₃ |
| 26 | 3 | —(CH₂)₄—SO₂CH₃ |
| 27 | 3 | —(CH₂)₅—SO₂CH₃ |
| 28 | 3 | —(CH₂)₆—SO₂CH₃ |
| 29 | 4 | —(CH₂)₄—SO₂CH₃ |
| 30 | 4 | —(CH₂)₅—SO₂CH₃ |
| 31 | 4 | —(CH₂)₆—SO₂CH₃ |
| 32 | 5 | —(CH₂)₄—SO₂CH₃ |
| 33 | 5 | —(CH₂)₅—SO₂CH₃ |
| 34 | 5 | —(CH₂)₆—SO₂CH₃ |
| 35 | 6 | —(CH₂)₄—SO₂CH₃ |
| 36 | 2 | 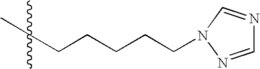 |
| 37 | 3 | 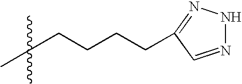 |
| 38 | 4 | 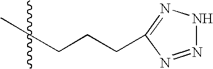 |
| 39 | 5 | 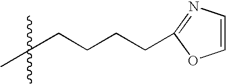 |
| 40 | 6 | 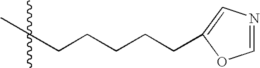 |
| 41 | 2 | 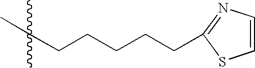 |
| 42 | 3 | 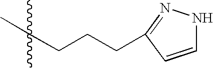 |
| 43 | 4 | 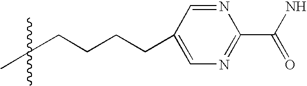 |
| 44 | 5 | 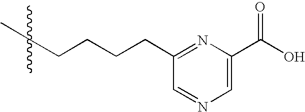 |
| 45 | 6 | 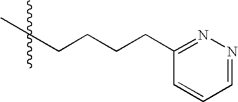 |
| 46 | 2 | 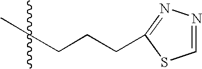 |
| 47 | 3 | 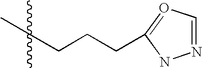 |
| 48 | 4 | —(CH₂)₃—NHSO₂CH₃ |
| 49 | 5 | —(CH₂)₄—NHSO₂CH₃ |
| and 50 | 6 | —(CH₂)₅—NHSO₂CH₃. |

17. The compound of claim 1 having the structural formula: 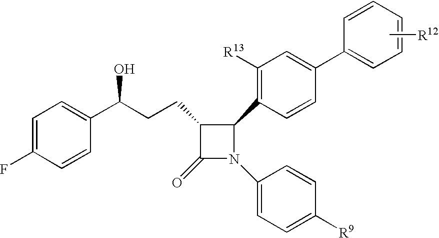
or a pharmaceutically acceptable salt thereof, wherein
R¹³ is selected from the group consisting of —H, —F, and —OH; and
R¹² and R⁹ are defined as follows:

| Compound No. | R¹² | R⁹ |
|---|---|---|
| 52 | 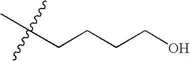 | 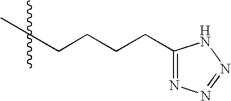 |
| 53 | 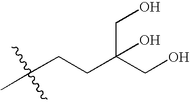 | 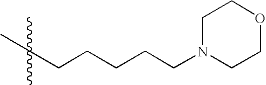 |
| 54 | 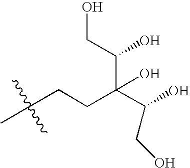 | 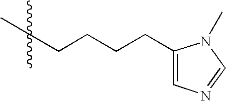 |
| 55 | 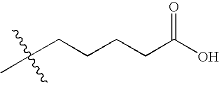 | 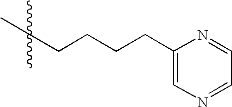 |
| 56 | 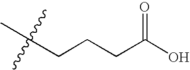 | 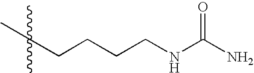 |
| 57 | 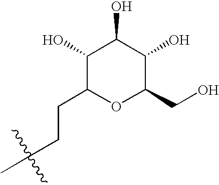 | 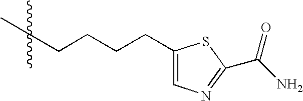 |
| and 58 | 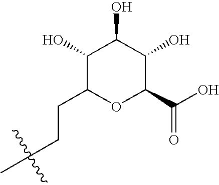 | 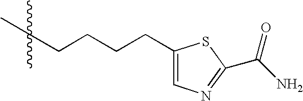 |

18. A method for lowering plasma LDL-cholesterol levels comprising administering a therapeutically effective amount of a compound of claim 1 to a patient in need of such treatment, optionally in combination with a therapeutically effective amount of at least one additional active agent selected from a lipid modifying agent, an anti-diabetic agent and an anti-obesity agent.

19. A method for reducing the risk for having an atherosclerotic disease event comprising administering a prophylactically effective amount of a compound of claim 1 to a patient at risk for such an event.

20. A pharmaceutical composition comprising a compound of claim 1 and a pharmaceutically acceptable carrier, and optionally comprising at least one additional active agent selected from a lipid modifying agent, an anti-diabetic agent and an anti-obesity agent.
